SEQUENCE LISTING (12 sequences)

SEQ ID NO: 1 - DNA, 1467 bp, Sudan virus
atggagggtctgtcattgctgcagctccctcgcgataagttccgtaaaagctcattcttcgtatgggtcataattctgttccagaaggcgttctctatgcccctcggcgtcgtgacaaacagtactttggaagtcacggaaatagaccagctcgtttgcaaggatcatttggcttcgacagaccagctgaaaagtgtgggcctcaatcttgaaggatcgggcgtgtcgaccgatatcccctccgctacgaaaagatggggtttccgcagcggtgttcccccaaaagtggtgagctatgaggctggcgagtgggctgaaaactgttacaatttggagattaagaagccagatggatcagagtgtctgcctccccctccagacggtgttaggggctttccgcgttgcagatacgtgcacaaagcccagggaactggaccgtgcccaggcgactatgctttccacaaagacggcgcctttttcctctacgacagattggcgtctaccgttatctaccgtggtgtcaacttcgctgagggagtaatcgctttccttatcctcgctaagccaaaggaaactttcctccaaagcccgcctatccgcgaagcggtcaactataccgagaatacttcctcctactacgccacctcttacttggagtacgagatagagaacttcggtgcacagcacagtactacactgttcaaaatcaataacaacacttttgtgttgctggatagaccccatacaccccaattccttttccagctcaacgacacgattcacttgcaccaacagttgtctaatacaactggaaaactcatttggactcttgatgcaaacatcaacgctgacatcggagaatgggccttttgggagaataagaagaacttgtccgagcaactgaggggtgaggaactctcattcgaaaccctttcgcttccccaagagagtacctcgaacggactcatcacctctaccgtaactggcatccttggttctcttggcctgcgcaagaggtcaagacgtcaggtcaatacccgcgctacgggcaaatgcaacccgaatctccattactggaccgcacaagaacagcacaacgcagcaggcatcgcctggatcccgtattttggaccaggtgccgagggtatctacacggagggtttgatgcacaaccaaaacgccctggtttgtggattgcgccaacttgcgaacgaaacgactcaggccttgcagctgtttctgcgcgcgactaccgaacttaggacatacacaatactgaaccgtaaggccattgacttcctgttgagaaggtggggtggtacctgtcgtatcttgggaccagattgctgcattgaacctcacgactggacgaagaacattacagacaagataaaccaaatcatccatgatttcatcgacaaccctctgccaaatcaccaccaccatcatcatcaccatcaccactga

SEQ ID NO: 2 - protein, 488 aa, Sudan virus
MEGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKINNNTFVLLDRPHTPQFLFQLNDTIHLHQQLSNTTGKLIWTLDANINADIGEWAFWENKKNLSEQLRGEELSFETLSLPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNHHHHHHHHHH

SEQ ID NO: 3 - DNA, 1491 bp, Ebola virus
atgggtgttacgggcatactgcagctgcctcgcgaccgtttcaagcgtacgtctttcttcttgtgggtcattatcctcttccagagaactttctcaattccactcggagtgatacataatagtactttgcaagtttccgacgtggacaagttggtttgtcgtgataaactgtcatcgaccaaccaactccgttctgttggactcaacctcgaaggcaacggtgtggcaactgacgtcccatccgccaccaaaaggtggggatttcgctccggagtgccccctaaagtggtcaattatgaagcaggtgaatgggctgagaattgctacaatttggagatcaagaaacccgacggatctgagtgccttccagcagcgcccgacggtatccgtggcttcccacgttgccgctacgtgcacaaggtcagtggcactggtccgtgcgctggcgacttcgctttccacaaggagggcgcatttttcctctacgaccgtcttgcatctaccgtcatctacagaggcacaacgttcgctgagggtgtggtagcgttcctgatattgccccaggctaagaaggacttcttcagtagccaccctttgcgcgagcctgtcaacgccactgaagatccttcatctggttactactcgactaccattaggtaccaggctacgggcttcggtaccaacgaaaccgagtacctcttcgaagtcgacaaccttacttatgtgcagctggagagccgcttcacgccccagtttctcttgcagctgaacgagacaatctacaccagcggaaagaggtccaacactaccggtaaactgatttggaaagtaaaccctgaaattgacactacaataggcgagtgggccttttgggagacaaagaagaatctgaccagaaagatccgcagtgaggaactgtcattcacggttgtcaacacccaccatcaagataccggtgaggaaagcgccagttcgggaaaattgggccttattacaaacacaatcgcgggtgtggcaggcctcatcaccggaggaaggcgtacaaggcgcgaggcgatcgtaaacgctcaacctaagtgcaatcccaacttgcactattggaccactcaagacgagggtgccgctatcggcctcgcctggataccctacttcggtccagctgcggaaggtatctatacagaaggactgatgcacaaccaagatggcttgatctgcggtctgcgccaactggcaaatgagacaacgcaggccttgcagcttttccttagagcgactaccgaactgaggacattttcaattcttaatagaaaggccatcgattttctgctccaacgctggggtggaacctgtcatatacttggcccggattgttgtatcgagccacacgactggactaagaacatcaccgacaagatagatcagatcattcacgatttcgtcgataaaacactgcctgaccaccaccatcaccatcatcatcaccaccattga

SEQ ID NO: 4 - protein, 496 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDHHHHHHHHHH

SEQ ID NO: 5 - DNA, 1434 bp, Marburg virus
atgccgctgtacaaactcttgaatgtgttgtggcttgtagcagtctccaatgcgttgcccatcctcgaaatcgcctccaacattcagccacagaatgtggactccgtgtgctctggaaccttgcaaaagacagaggacgtccacctcatgggattcacgctgtctggacaaaaggtagcggactcgcctttggaagcatctaagaggtgggcttttcgtgctggagtacccccaaagaacgtcgaatacacggagggtgaggaggcgaaaacttgctacaacattagtgttaccgatccgagtggcaaatcattgcttcttgaccctcccactaatatccgtgattaccccaaatgcaaaactatccaccacattcagggccaaaatccgcatgctcagggaatcgccctgcacttgtggggtgcattcttcctctatgatcgcatagcctctacaaccatgtacagaggtaaagtgttcacggaaggcaatatcgccgctatgatcgtcaacaagacggtgcataagatgattttcagccgccagggtcaaggttaccgccacatgaatctgacgtcaaccaacaagtattggacttcgagcaacggaacacagacaaacgataccggttgtttcggcactttgcaagaatacaatagtaccaaaaaccagacgtgcgccccatctaaaaagcccctcccgcttcctactgcgcaccccgaggtcaagctgacttccacctccacggacgctacaaagctcaacactactgacccaaatagtgacgattccacggcccaacacctggttagccgtagaaggaagaggaatatcttgtggagagagggtgacatgttccctttcctcgacggactcatcaatgcgccgattgactttgaccctgtacctaatacgaaaaccatcttcgacgaaagttctagctccggtgcctcagccgaggaagatcagcacgcttcccctaacatatccctgactctgtcctattttccgaaagtcaacgaaaatacagcccactccggcgaaaacgagaatgattgtgatgctgagctgaggatctggagcgtccaagaggatgatctggcagccggactcagttggatacctttcttcggtcctggaatcgagggcctgtacactgctggtttgattaagaaccagaacaacttggtttgccgccttagacgcctcgccaaccaaacggcaaagagtctggagcttctcctccgcgtgaccactgaagaaaggacattcagtctgattaacagacatgccatcgactttttgttggctcgttggggcggaacttgtaaggtcctcggacctgactgttgcatcggtattgaagatctgtcacgtaatatcagcgagcaaatagaccaaatcaagaaggatgagcagaaggaaggcacctga

SEQ ID NO: 6 - protein, 477 aa, Marburg virus
MPLYKLLNVLWLVAVSNALPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTDPNSDDSTAQHLVSRRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGT

SEQ ID NO: 7 - DNA, 1944 bp, Sudan virus
atggagggattgtcgcttttgcagttgccaagagacaagtttcgcaaatcgtcctttttcgtctgggttatcatcctcttccaaaaggccttctcaatgcctctgggcgttgttaccaactcaactctggaagtaaccgagattgaccagctcgtgtgcaaagaccacttggcctccaccgaccagctcaaatcagttggactcaatctcgaaggtagtggagtgagcacagacataccaagtgcgactaagaggtggggtttccgtactggagtacctcccaaagttgtctcgtacgaagctggcgaatgggcggaaaactgttacaacttggagattaagaaacccgacggttctgaatgcctcccgcctccgcctgatggtgtgcgcggtttccctcgttgcagatatgtgcacaaagcacagggaacaggcccctgccctggcgattacgctttccataaagacggagccttctttctttatgaccgtttggctagtacggttatctacagaggtgtcaattttgctgaaggtgtcatcgcgttcctgatcctggctaagccgaaggagactttccttcaatcgcctcctatccgtgaggctgtcaattacaccgaaaacacgagttcatactacgctacctcatatctcgaatacgagatcgagaactttggcgcacagcattctactaccctcttcaaaatcaataacaatactttcgtgctgctcgatagaccgcacactccccaattcttgtttcaactcaacgatacgatccatttgcatcagcaattgagcaatactacgggaaagctgatttggaccttggacgctaacattaacgctgatatcggagagtgggcattttgggagaacaagaagaatttgtccgagcagctgcgcggtgaagaactgtctttcgagacactgtctctgaacgagacagaggacgacgacgccacatccagcagaacaaccaagggtcgcatctcagacagggccactagaaagtactcagatctggtccccaaagactctccgggaatggtgtcactgcacgtgcccgaaggcgaaactactttgccatctcaaaactccaccgaaggtcgtagagtcgacgtgaacactcaagaaactattactgaaacgaccgcgacgatcataggcaccaacggaaacaacatgcagatctcgacaatcggtactggcctgagctcatcccaaatactcagctctagccccacaatggccccgagtcccgagactcagacttctaccacctatacccccaagctcccagtgatgactactgaagaatccactaccccaccacgcaacagtcctggctccacaaccgaggctccaaccttgactactccagaaaacattactacagctgtcaagaccgtgttgccgcaagagagtacatcgaatggcctgataacttccaccgtgaccggtattcttggtagtctcggcctccgtaagagaagccgtcgccaagtaaacacacgcgcaacaggaaaatgtaaccctaatttgcattactggaccgcgcaagaacagcacaacgctgccggcatcgcgtggattccatacttcggtcccggtgccgagggaatctacactgaaggcttgatgcacaatcagaacgcacttgtttgtggcctccgccaactggccaatgagactacccaggctctgcagttgtttctgcgtgcaacgacggaacttaggacatacactatattgaaccgcaaggccattgacttcttgctcaggcgttggggcggtacgtgcagaatacttggcccagactgttgcatagagccacacgattggaccaagaacatcacggataagatcaaccagatcatccacgatttcatcgataacccccttcccaaccaccatcaccatcatcaccaccaccatcactga

SEQ ID NO: 8 - protein, 647 aa, Sudan virus
MEGLSLLQLPRDKFRKSSFFVWVIILFQKAFSMPLGVVTNSTLEVTEIDQLVCKDHLASTDQLKSVGLNLEGSGVSTDIPSATKRWGFRTGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKINNNTFVLLDRPHTPQFLFQLNDTIHLHQQLSNTTGKLIWTLDANINADIGEWAFWENKKNLSEQLRGEELSFETLSLNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEESTTPPRNSPGSTTEAPTLTTPENITTAVKTVLPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNHHHHHHHHHH

SEQ ID NO: 9 - DNA, 1944 bp, Ebola virus
atgggagttacgggcatattgcagctgcctagagacagattcaagaggacttccttcttcttgtgggtaatcatcttgttccagaggactttttcaatcccgttgggcgttattcataactctactctgcaagtgtccgatgtcgataagctcgtatgtagggacaagctcagctcaacaaaccagttgcgttccgtgggcctgaacctcgagggaaatggagtagccaccgatgtaccctctgcaaccaagaggtggggcttccgcagcggtgtccctcccaaagtcgtgaattacgaagctggcgaatgggccgagaattgctataacctcgagatcaagaagccggacggctctgaatgtctgcctgccgctcctgacggtattcgcggtttcccacgttgccgttacgtgcataaagtgtccggtacgggaccatgcgcaggcgactttgcgttccacaaagagggagcctttttcctctacgaccgcctggcttcgactgtcatctatcgtggaaccacattcgccgaaggtgtggtagcctttctgatcctgccacaggccaagaaggacttcttctctagccacccactgagggaaccagtcaacgctacagaagatcccagcagtggttactacagtactaccatccgctaccaagcgaccggtttcggaacgaatgagactgagtatctctttgaggtggataacctgacctacgtgcaactcgaatccagattcacgcctcaatttcttctccaattgaacgagactatctacacttccggaaaacgtagtaacactacgggaaagctgatttggaaggtgaaccccgagatcgatacgactattggcgagtgggccttttgggaaacaaagaaaaacttgacgaggaagattcgctctgaagaactgtccttcactgtcgtgtcaaacggagcgaaaaacatttcgggccagagtccagcccgcacctcgagtgatcccggtaccaatacaactacagaggaccataagatcatggcttcagagaactcctcggccatggtccaagtccacagtcaaggacgcgaagctgcagttagccacctgactacactggcgaccataagcacatctccgcagagccttacgacaaaacctggtcccgacaactctacgcataatacgcccgtgtacaaactcgatattagcgaagccacccaagtcgaacagcaccaccgtagaactgacaacgactcaactgcaagtgataccccttcggccaccacagctgctggcccacctaaagctgagaacaccaatactagcaaatcgaccgatttcctcgaccctgctacaaccacatcacctcagaatcactccgaaacagcgggtaacaataacacccaccatcaagacacgggcgaggaatctgcctcttccggcaaactcggtcttatcacaaacactatcgcgggtgtcgctggattgatcaccggtggtaggcgcactcgtagagaggcaatcgttaatgcccagccaaagtgtaaccccaacttgcattattggactactcaagatgagggagcagctataggactcgcttggatcccttacttcggacccgctgcagagggcatctacacggaaggtttgatgcacaaccaggacggccttatttgcggtctgaggcagcttgctaacgagacaacgcaagccctccaactgttcctccgcgcgacgacggagttgcgcaccttcagtattctcaacaggaaggctattgatttcttgttgcagcgctggggtggtacctgccacatcttgggcccagactgttgcatcgagccccacgattggactaagaacattaccgacaagatcgaccagatcatccacgattttgttgacaagacgttgccagaccatcaccatcatcatcaccaccaccatcactga

SEQ ID NO: 10 - protein, 647 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDHHHHHHHHHH

SEQ ID NO: 11 - DNA, 1918 bp, Marburg virus
atgccgctgtacaaactcttgaatgtgttgtggcttgtagcagtctccaatgcgttgcccatcctcgaaatcgcctccaacattcagccacagaatgtggactccgtgtgctctggaaccttgcaaaagacagaggacgtccacctcatgggattcacgctgtctggacaaaaggtagcggactcgcctttggaagcatctaagaggtgggcttttcgtgctggagtacccccaaagaacgtcgaatacacggagggtgaggaggcgaaaacttgctacaacattagtgttaccgatccgagtggcaaatcattgcttcttgaccctcccactaatatccgtgattaccccaaatgcaaaactatccaccacattcagggccaaaatccgcatgctcagggaatcgccctgcacttgtggggtgcattcttcctctatgatcgcatagcctctacaaccatgtacagaggtaaagtgttcacggaaggcaatatcgccgctatgatcgtcaacaagacggtgcataagatgattttcagccgccagggtcaaggttaccgccacatgaatctgacgtcaaccaacaagtattggacttcgagcaacggaacacagacaaacgataccggttgtttcggcactttgcaagaatacaatagtaccaaaaaccagacgtgcgccccatctaaaaagcccctcccgcttcctactgcgcaccccgaggtcaagctgacttccacctccacggacgctacaaagctcaacactactgacccaaatagtgacgatgaggacttgactacatcgggttcgggctctggtgagcaggagccctataccacgagcgacgctgccaccaaacaaggcctgagctcaaccatgccccctactccatcacctcaaccctcaacccctcagcagggaggtaacaacacgaaccatagtcaaggcgtggttaccgaacccggaaagaccaacacaaccgcccagccaagtatgccaccgcataacacgaccacgatttctacaaacaacacctccaaacacaaccttagcactccctccgttccaatccagaacgccacgaactacaatactcaatctacagctccagaaaacgaacagacctctgcaccaagtaagactacccttctccctacggagaaccccaccaccgcaaagtccactaactcgacaaagtcgcccacaacgactgtgccaaataccacaaacaagtactcaacttctccctctccgacacctaactccacggcccaacacctggttagccgtagaaggaagaggaatatcttgtggagagagggtgacatgttccctttcctcgacggactcatcaatgcgccgattgactttgaccctgtacctaatacgaaaaccatcttcgacgaaagttctagctccggtgcctcagccgaggaagatcagcacgcttcccctaacatatccctgactctgtcctattttccgaaagtcaacgaaaatacagcccactccggcgaaaacgagaatgattgtgatgctgagctgaggatctggagcgtccaagaggatgatctggcagccggactcagttggatacctttcttcggtcctggaatcgagggcctgtacactgctggtttgattaagaaccagaacaacttggtttgccgccttagacgcctcgccaaccaaacggcaaagagtctggagcttctcctccgcgtgaccactgaagaaaggacattcagtctgattaacagacatgccatcgactttttgttggctcgttggggcggaacttgtaaggtcctcggacctgactgttgcatcggtattgaagatctgtcacgtaatatcagcgagcaaatagaccaaatcaagaaggatgagcagaaggaaggcacctgataagctt

SEQ ID NO: 12 - protein, 636 aa, Marburg virus
MPLYKLLNVLWLVAVSNALPILEIASNIQPQNVDSVCSGTLQKTEDVHLMGFTLSGQKVADSPLEASKRWAFRAGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKKPLPLPTAHPEVKLTSTSTDATKLNTTDPNSDDEDLTTSGSGSGEQEPYTTSDAATKQGLSSTMPPTPSPQPSTPQQGGNNTNHSQGVVTEPGKTNTTAQPSMPPHNTTTISTNNTSKHNLSTPSVPIQNATNYNTQSTAPENEQTSAPSKTTLLPTENPTTAKSTNSTKSPTTTVPNTTNKYSTSPSPTPNSTAQHLVSRRRKRNILWREGDMFPFLDGLINAPIDFDPVPNTKTIFDESSSSGASAEEDQHASPNISLTLSYFPKVNENTAHSGENENDCDAELRIWSVQEDDLAAGLSWIPFFGPGIEGLYTAGLIKNQNNLVCRLRRLANQTAKSLELLLRVTTEERTFSLINRHAIDFLLARWGGTCKVLGPDCCIGIEDLSRNISEQIDQIKKDEQKEGT

(19) United States
(12) Patent Application Publication
(10) Pub. No.: US 2018/0022790 A1
(43) Pub. Date: Jan. 25, 2018

(54) EQUINE IMMUNOGLOBULIN COMPOSITIONS AND USES FOR TREATING FILOVIRUS-MEDIATED DISEASES

(71) Applicants: INTEGRATED BIOTHERAPEUTICS, INC., Gaithersburg, MD (US); AUBURN UNIVERSITY, Auburn University, AL (US)

(72) Inventors: Frederick Wayne HOLTSBERG, Taneytown, MD (US); Mohammad Javad AMAN, Rockville, MD (US); Paul H. WALZ, Auburn, AL (US); Stephanie R. OSTROWSKI, Auburn, AL (US)

(21) Appl. No.: 15/547,379

(22) PCT Filed: Jan. 28, 2016
(86) PCT No.: PCT/US2016/015257

§ 371 Date: Jul. 28, 2017

Related U.S. Application Data
(60) Provisional application No. 62/109,042, filed on Jan. 28, 2015.

(51) Int. Cl. C07K 16/10

(52) U.S. Cl. CPC C07K 16/10; C07K 2317/20; A61K 2039/505

(57) ABSTRACT

The disclosure provides a composition comprising immunoglobulin from an equine that has been hyper-immunized with a filovirus immunogen. The disclosure further provides methods of making such compositions and methods of using such compositions, e.g., for treating Ebola virus infection.

### CROSS-REFERENCE

This application claims the benefit of and priority to U.S. Provisional Application No. 62/109,042, filed Jan. 28, 2015, which is incorporated by reference herein in its entirety.

### INCORPORATION OF SEQUENCE LISTING

The sequence listing that is contained in the file named “57883_152630_PCT Seq List_ST25.txt”, which is 43,534 bytes (measured in operating system MS-Windows), created on Jan. 27, 2016, is filed herewith by electronic submission and incorporated herein by reference in its entirety.

### BACKGROUND

The filoviruses Ebola (EBOV) and Marburg (MARV) are two of the most pathogenic viruses in humans and non-human primates (Feldman and Klenk, Adv. Virus Res. 47:1 (1996), which cause a severe hemorrhagic fever (Johnson et al., Lancet 1:569 (1997)). The main Filovirus species causing outbreaks in humans are Ebola viruses Zaire (EBOV) and Sudan virus (SUDV), as well as the Lake Victoria MARV species. The mortality rates associated with infections of Ebola or Marburg virus are up to 90% (Feldman and Klenk, 1996, supra; Johnson et al., 1997, supra). The lack of immunological and pharmacological therapeutic measures, poses a challenge to classification of the public health threat of Marburg and Ebola viruses.

Filoviruses are enveloped, single-stranded, negative sense RNA filamentous viruses and encode seven proteins, of which the spike envelope glycoprotein (GP) is considered the main protective antigen. Filovirus, e.g., EBOV or MARV GP is proteolytically cleaved by furin protease into two subunits linked by a disulfide linkage: GP1 (˜140 kDa) and GP2 (˜38 kDa) (Manicassamy, B., J. et al. J Virol. 79:4793-805 (2005)). Three GP1-GP2 units form the trimeric GP envelope spike (˜550 kDa) on the viral surface (Feldmann, H. et al. Arch Virol Suppl 7:81-100 (1993); Feldmann, H. et al. Virology 182:353-6 (1991); Geisbert, T. W., and P. B. Jahrling. Virus Res 39:129-50 (1995); Kiley, M. P. et al. J Gen Virol 69:1957-67 (1988)). GP1 mediates cellular attachment (Kiley, M. P. et al. (1988); Kuhn, J. H. et al. J Biol Chem 281:15951-8 (2006)), and contains a mucin-like domain which is variable in sequence, heavily glycosylated and has little or no predicted secondary structure (Sanchez, A. et al. J Virol 72:6442-7 (1998)).

The primary protective antigen of EBOV is the envelope glycoprotein (GP) (Marzi, A. and H. Feldmann, Expert Rev Vaccines, 2014. 13(4): p. 521-31). A series of recent reports indicate that antibodies targeting GP alone can provide significant protection against Ebola hemorrhagic fever.

Dye et al showed that purified convalescent IgG from macaques can protect non-human primates (NHPs) against challenge with Marburg virus (MARV) and EBOV when administered as late as 48 h post exposure (Dye, J. M., et al., Proc Natl Acad Sci USA, 2012). Olinger et al reported significant protection from Zaire EBOV (ZEBOV) challenge in NHPs treated with a cocktail of three monoclonal antibodies (mAbs) to GP (MB-003: 6D8, 13C6, 13F6) administered 24 h or 48 h post exposure (Olinger, G. G., Jr., et al., Proc Natl Acad Sci USA, 2012. 109(44): p. 18030-5). Administration of MB-003 after emergence of symptoms in EBOV infected rhesus macaques also led to 43% protection (Pettitt, J., et al., Sci Transl Med, 2013. 5(199): p. 199ra113). Recent results published from Kobinger lab showed that a different antibody combination (ZMab: 1H3, 2G4, 4G7) was protective at 24 h or 48 h post challenge (Qiu, X., et al., Sci Transl Med, 2012. 4(138): p. 138ra81) and this resulted in sustained protective immune response for at least 10 weeks (Qiu, X., et al., Sci Rep, 2013. 3: p. 3365). Combination of ZMab with adenovirus-expressed IFNα protected macaques when administered after animals were symptomatic (Qiu, X., et al., Sci Transl Med, 2013. 5(207): p. 207ra143). Further studies aimed at optimizing the best treatment combination revealed that a combination of the mAbs 13C6 (from MB-003) with 2G4 and 4G7 (from ZMab) provided full protection as late as 5 days post challenge in macaques (Qiu, X., et al., Nature, 2014. 514(7520):47-53). This combination, coined as ZMapp, was also given to at least seven individuals infected with EBOV in the current outbreak with anecdotal indications of success.

Virus-like proteins (VLP) vaccines have been generated based on sequences from three major species of filoviruses (Ou et al., J Virol. 9:32 (2012)). Formation of filovirus VLPs is described in Bavari, S., et al, J. Exp. Med. 195:593-602 (2002). The VLPs were formed by expression of two viral proteins GP and VP40, denoted here as Double VLP. Double VLPs exhibited protective efficacy in mice (Warfield, K. L., et al. Proc Natl Acad Sci USA. 100:15889-94 (2003)). VLPs can be also produced with three viral proteins GP, VP40, and NP, which increases the yield and stability of the VLPs (Kallstrom, G., et al. J Virol Methods. 127(1):1-9 (2005)).

Role of Antibodies in Protection Against Filovirus Hemorrhagic Fever.

While both T and B cell responses are reported to play a role in protective immune responses to filoviruses (Warfield, et al., 2005, J Immunol, 175 (2):1184-1191), a series of recent reports indicate that antibody alone can provide protection. Dye et al showed that purified convalescent IgG from macaques can protect non-human primates (NHPs) against challenge with MARV and EBOV when administered as late as 48 h post exposure (Dye, et al., 2012, Proc Natl Acad Sci USA, 109(13):5034-9). Olinger et al reported protection from EBOV challenge in NHPs treated with a cocktail of three monoclonal antibodies (mAbs) to GP administered 24 h and 48 h post exposure (Olinger, et al., 2012, Proc Natl Acad Sci USA, 109 (44):18030-18035). Similar results were also reported in two other studies (Qiu, et al., 2013, Sci Transl Med, 5 (207):207ra143; Qiu, et al., 2013, J Virol, 87 (13):7754-7757). Collectively these data demonstrate that a humoral response can control, alleviate, reduce, or prevent, filovirus infection.

Equine Immunoglobulin for Use in Human Therapeutics:

Hyperimmune antibody preparations from horse serum or plasma have been used over the past century for the treatment of humans suffering from a variety of infectious diseases, intoxication, or envenomation. Specific infectious agents and medical emergencies where equine-origin hyperimmune plasma and/or derivatives have been utilized include snake envenomation (Theakston, R. D. and D. A. Warrell, Toxicon, 1991. 29(12): p. 1419-70), spider bites (Dart, R. C., et al., Aim Emerg Med, 2013. 61(4): p. 458-67), botulism (Fagan, R. P., et al., Clin Infect Dis, 2011. 53(9): p. e125-8; Hill, S. E., et al., Aim Pharmacother, 2013. 47(2): p. e12), rabies (Goudsmit, J., et al., J Infect Dis, 2006. 193(6): p. 796-801), diphtheria (Peter, G., Pediatrics, 1997. 100(1): p. 109-11), and tetanus (Kabura, L., et al., Trop Med Int Health, 2006. 11(7): p. 1075-81). In addition, an equine-origin anti-thymocyte globulin (Atgam®, Pfizer) has been developed and used clinically under stringent guidelines in renal transplant patients for the management of allograft rejection and in patients with aplastic anemia (Malhotra, P., et al., Hematology, 2014).

The horse as the source of hyperimmune IgG has tremendous advantage in terms of offering a high-yield, low-cost source of antibodies for use in human therapeutics. In the past, equine antisera have been associated with adverse reactions and serum sickness. Current processing techniques to generate Fab or F(ab′)₂ components greatly reduce the complications associated with use of equine-origin hyperimmune antibody therapy utilizing whole IgG. In a recent clinical trial involving scorpion antivenom comprised of an equine origin F(ab′)₂, rates of immune reaction were two orders of magnitude lower than the range of reactions historically reported with use of minimally refined whole IgG products (Boyer, L., et al., Toxicon, 2013. 76: p. 386-93).

There remains a need for effective and economical treatments for diseases caused by filoviruses. This disclosure provides equine immunoglobulin with high titer of antibodies directed against protective epitopes of EBOV or MARV GP that can provide effective protection against filovirus-mediated disease, including Ebola hemorrhagic fever.

### SUMMARY

The present disclosure provides for a pharmaceutical composition comprising polyclonal immunoglobulin from an equine that has been hyper-immunized with a filovirus glycoprotein. In certain embodiments, the immunoglobulin is purified from serum or plasma of the equine that has been hyper-immunized with the filovirus glycoprotein. In certain embodiments, the purified immunoglobulin is IgG, or a fragment thereof. In certain embodiments, at least 1%, 2%, 3%, 4%, 5%, 6%, 7%, 8%, 9%, 10%, 11%, 12%, 13%, 14%, 15%, 20%, or more of the purified IgG binds to the filovirus glycoprotein.

In some embodiments, the purified immunoglobulin can prevent or minimize symptoms in a subject infected with a filovirus. In certain embodiments, the filovirus is Ebola virus (EBOV), Sudan virus (SUDV), Bundibugyo virus (BDBV), Tai Forrest virus (TAFV), Reston virus (RESTV), or Marburg virus (MARV).

In some embodiments, the equine is immunized with a mucin-like domain-deleted filovirus spike glycoprotein. In some embodiments, the transmembrane domain of the spike glycoprotein is deleted. In certain embodiments, the spike glycoprotein comprises the GP1 subunit or a fragment thereof from MARV, EBOV, SUDV, BDBV, TAFV, RESTV, or a combination of GP1 subunits thereof. In certain embodiments, the spike glycoprotein comprises the GP1 subunit or a fragment thereof and the GP2 subunit or a fragment thereof from MARV, EBOV, SUDV, BDBV, TAFV, RESTV, or a combination of GP1 and GP2 subunits thereof. In certain embodiments, the spike glycoprotein comprises an amino acid sequence that is at least 90%, at least 95%, or 100% identical to SEQ ID NOs: 2, 4, 6, 8, 10, or 12.

In some embodiments, the equine is immunized with the spike glycoprotein on days 0, 21, 42, and 63. In some embodiments, the polyclonal immunoglobulin is recovered as plasma on day 90 via plasmapheresis. In certain embodiments, the immunogen comprises MARV GP-ΔTM and the recovered plasma has an EC50 titer for binding to MARV GP-ΔTM of at least 10², at least 5×10², at least 10³, at least 5×10³, least 10⁴, at least 5×10⁴, least 10⁵, or at least 5×10⁵, as determined by ELISA. In certain embodiments, the immunogen comprises MARV GP-ΔTM and the purified IgG binds to MARV GP-ΔTM with an EC50 of less than 3 μg/ml, less than 2.5 μg/ml, less than 2 μg/ml, less than 1.5 μg/ml, or less than 1 μg/ml, or less than 0.5 μg/ml, as measured ELISA.

In some embodiments, two doses of about 100 mg/kg administered to a mouse following a lethal challenge with a filovirus can protect the mouse against the lethal challenge.

In some embodiments, the equine is immunized in a prime-boost regimen. In certain embodiments, the prime-boost regimen comprises priming with a filovirus virus-like particle (VLP) and boosting with the spike glycoprotein. In certain embodiments, the VLP comprises a filovirus glycoprotein and a filovirus VP40. In certain embodiments, the VLP further comprises the filovirus nucleoprotein (NP). In certain embodiments, the prime dose is administered on day zero and day 21, and the boost dose is administered on day 42 and day 63. In certain embodiments, the polyclonal immunoglobulin is recovered as plasma on day 90 via plasmapheresis.

In some embodiments, the priming immunogen comprises an EBOV VLP comprising GP, VP40, and NP, the boosting immunogen comprises EBOV GP-ΔMuc, and the recovered plasma has an EC50 titer for binding to EBOV GP-ΔTM of at least 10³, at least 5×10³, least 10⁴, at least 5×10⁴, least 10⁵, or at least 5×10⁵, as determined by ELISA. In some embodiments, the priming immunogen comprises an EBOV VLP comprising GP, VP40, and NP, the boosting immunogen comprises EBOV GP-ΔMuc, and the purified IgG binds to EBOV GP-ΔTM or EBOV GP-ΔMuc with an EC50 of less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, less than 0.1 μg/ml, or less than 0.09 μg/ml, as measured ELISA.

In some embodiments, two doses of about 100 mg/kg administered to a mouse following a lethal challenge with a filovirus can protect the mouse against the lethal challenge.

The present disclosure also provides for a method of preparing any of the composition disclosed herein. In certain embodiments, a method comprises administering an amount of a filovirus immunogen to an equine sufficient to hyperimmunize the equine against protective antigens of the filovirus, where the immunogen comprises a filovirus spike glycoprotein; and recovering immunoglobulin from the equine. In certain embodiments, the immunoglobulin is recovered as plasma.

In some embodiments, the method further comprises purifying the immunoglobulin recovered from the equine. In certain embodiments, the purified immunoglobulin comprises IgG or a fragment thereof.

The present disclosure also provides a method of preventing, treating, or managing a filovirus-mediated disease in a subject where the method comprises administering to a subject in need of treatment a polyclonal immunoglobulin from an equine that has been hyper-immunized with a filovirus glycoprotein. In certain embodiments, the immunoglobulin is purified from serum or plasma of the equine that has been hyper-immunized with the filovirus glycoprotein. In certain embodiments, the purified immunoglobulin is IgG, or a fragment thereof. In certain embodiments, at least 1%, 2%, 3%, 4%, 5%, 6%, 7%, 8%, 9%, 10%, 11%, 12%, 13%, 14%, 15%, 20%, or more of the purified IgG binds to the filovirus glycoprotein.

In some embodiments, the purified immunoglobulin can prevent or minimize symptoms in a subject infected with a filovirus. In certain embodiments, the filovirus is MARV, EBOV, SUDV, BDBV, TAFV, or RESTV.

In some embodiments, the equine is immunized with a mucin-like domain-deleted filovirus spike glycoprotein. In certain embodiments, the transmembrane domain of the spike glycoprotein is deleted. In certain embodiments, the spike glycoprotein comprises the GP1 subunit or a fragment thereof from MARV, EBOV, SUDV, BDBV, TAFV, RESTV, or a combination of GP1 subunits thereof. In certain embodiments, the spike glycoprotein comprises the GP1 subunit or a fragment thereof and the GP2 subunit or a fragment thereof from MARV, EBOV, SUDV, BDBV, TAFV, RESTV, or a combination of GP1 and GP2 subunits thereof. In certain embodiments, the spike glycoprotein comprises an amino acid sequence that is at least 90%, at least 95%, or 100% identical to SEQ ID NOS: 2, 4, 6, 8, 10, or 12.

In some embodiments, the equine is immunized with the spike glycoprotein on days 0, 21, 42, and 63. In certain embodiments, the polyclonal immunoglobulin is recovered as plasma on day 90 via plasmapheresis.

In some embodiments, the immunogen comprises MARV GP-ΔTM and the recovered plasma has an EC50 titer for binding to MARV GP-ΔTM of at least 10², at least 5×10², at least 10³, at least 5×10³, least 10⁴, at least 5×10⁴, least 10⁵, or at least 5×10⁵, as determined by ELISA. In some embodiments, the immunogen comprises MARV GP-ΔTM and the purified IgG binds to MARV GP-ΔTM with an EC50 of less than 3 μg/ml, less than 2.5 μg/ml, less than 2 μg/ml, less than 1.5 μg/ml, or less than 1 μg/ml, or less than 0.5 μg/ml, as measured ELISA.

In some embodiments, two doses of about 100 mg/kg administered to a mouse following a lethal challenge with a filovirus can protect the mouse against the lethal challenge.

In some embodiments, the equine is immunized in a prime-boost regimen. In certain embodiments, the prime-boost regimen comprises priming with a filovirus VLP and boosting with the spike glycoprotein. In certain embodiments, the VLP comprises a filovirus glycoprotein and a filovirus VP40. In certain embodiments, the VLP further comprises the filovirus nucleoprotein (NP). In certain embodiments, the prime dose is administered on day zero and day 21, and the boost dose is administered on day 42 and day 63.

In some embodiments, the polyclonal immunoglobulin is recovered as plasma on day 90 via plasmapheresis.

In some embodiments, the priming immunogen comprises an EBOV VLP comprising GP, VP40, and NP, the boosting immunogen comprises EBOV GP-ΔMuc, and the recovered plasma has an EC50 titer for binding to EBOV GP-ΔTM of at least 10³, at least 5×10³, least 10⁴, at least 5×10⁴, least 10⁵, or at least 5×10⁵, as determined by ELISA. In some embodiments, the priming immunogen comprises an EBOV VLP comprising GP, VP40, and NP, the boosting immunogen comprises EBOV GP-ΔMuc, and the purified IgG binds to EBOV GP-ΔTM or EBOV GP-ΔMuc with an EC50 of less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, less than 0.1 μg/ml, or less than 0.09 μg/ml, as measured ELISA.

In some embodiments, two doses of about 100 mg/kg administered to a mouse following a lethal challenge with a filovirus can protect the mouse against the lethal challenge.

In some embodiments, the filovirus-mediated disease comprises one or more symptoms selected from the group consisting of: fever, internal hemorrhaging, edema, organ failure, headache, malaise, myalgia, nausea, vomiting, bleeding of needle puncture sites, hematemesis, melena, petechiae, ecchymosis, maculopapular rash, disseminated intravascular coagulation, shock, jaundice, conjunctivitis, diarrhea, pharyngitis, convulsions, delirium, coma, oligura, and epistaxis. In certain embodiments, the subject is a human.

### BRIEF DESCRIPTION OF THE DRAWINGS/FIGURES

FIG. 1: Domain structure of a filovirus glycoprotein. The abbreviation SP refers to signal peptide, and TM refers to transmembrane domain.

FIG. 2: Schematic of a filovirus GP-ΔMuc construct.

FIG. 3A-B: SDS-PAGE (A) and a Western Blot (B) of purified EBOV GP-ΔTM and GP-ΔMuc. In the reduced lanes, the top band is GPnull, middle is GP1, and lower is GP2. Lane 1: GP-ΔTM (reducing), Lane 2: GP-ΔTM (non-reducing), Lane 3: GP-ΔMuc (reducing), and Lane 4: GP-ΔMuc (non-reducing).

FIG. 4: Vaccination and plasmapheresis schedule of horses EBOV400-407.

FIG. 5A-B: Antibody response to EBOV GP-ΔTM (A) or GP-ΔMuc (B) in plasma of horses immunized with EBOV antigens.

FIG. 6A-B: Purified E401 IgG quality control. A) SDS-PAGE analysis. B) ELISA using purified GP-ΔTM of EBOV (upright triangle, left-most curve), Sudan (square, middle curve), or MARV (upside down triangle, right-most curve).

FIG. 7A-B: Mouse efficacy study results: (A) % weight change, (B) survival.

FIG. 8: Vaccination and plasmapheresis schedule of horses M300, M304, M305, M306.

FIG. 9A-B: Antibody response to MARV GP-ΔTM (A) or GP-ΔMuc (B) in plasma of horses immunized with MARV GP-ΔTM antigen.

FIG. 10A-B: Purified M304 IgG quality control. A) SDS-PAGE analysis. B) ELISA using purified GP-ΔTM of MARV (upside down triangle, left-most curve), SUDV (square, middle curve), or EBOV (upright triangle, right-most curve).

FIG. 11: Neutralization of MARV by IgG from MARV GP immunized horses at 1 mg/ml.

FIG. 12: Equine IgG mediated protection of mice from lethal MARV challenge.

### DETAILED DESCRIPTION

### Definitions

The term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

It is to be noted that the term “a” or “an” entity refers to one or more of that entity. As such, the terms “a” (or “an”), “one or more,” and “at least one” can be used interchangeably herein.

By a “filovirus” is meant a virus belonging to the family Filoviridae. Exemplary filoviruses are Ebola virus and Marburg virus. The virions of filoviruses contain seven proteins which include a surface glycoprotein (GP), a nucleoprotein (NP), an RNA-dependent RNA polymerase (L), and four virion structural proteins (VP24, VP30, VP35, and VP40).

By “subunit vaccine” is meant a vaccine produced from specific protein subunits of a virus and thus having less risk of adverse reactions than whole virus vaccines.

By the term “immunogen,” as used herein, is meant a composition comprising an antigen which, when inoculated into a mammal, has the effect of stimulating an immune response, e.g., a humoral immune response resulting in antibody production. A B-cell response results in the production of antibody that binds to the antigen. The vaccine can serve to elicit an immune response in the mammal which serves to protect the mammal against a disease.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology, but is not necessarily translated from a designated nucleic acid sequence. It can be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides that retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions that do not eliminate antigen-binding are well-known in the art (see, e.g., Brummell et al., Biochem. 32:1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids, and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, that has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

As used herein, a “coding region” is a portion of nucleic acid that consists of codons translated into amino acids. Although a “stop codon” (nucleic acid bases TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions that encode secretory or signal peptides, which direct the secretion of a polypeptide encoded by a polynucleotide as disclosed herein. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that cans specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

The terms “antibody” and “immunoglobulin” can be used interchangeably herein. An antibody (or a fragment, variant, or derivative thereof as disclosed herein) includes at least the variable domain of a heavy chain or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019 Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “subject” or “individual” or “host” or “patient,” which terms are used interchangeably herein, is meant any subject, particularly a mammalian subject, for whom prophylaxis or therapy is desired, particularly humans. Other subjects can include non-human primates, cattle, dogs, cats, guinea pigs, rabbits, rats, mice, horses, and so on.

As used herein, the terms “treatment”, “treating”, and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject predisposed to the disease or susceptible to the disease, but has not yet been diagnosed as having it (e.g., where the subject is susceptible to infection by a pathogen, but has not yet been infected by the pathogen), including, but not limited to, reducing the risk of disease and/or death following infection by a filovirus; reducing the incidence of disease and/or death following infection by a filovirus; reducing the incidence or risk of infection by a filovirus; and reducing the extent of disease following infection by a filovirus; (b) inhibiting the disease, i.e., arresting its development, slowing its progression; and (c) relieving the disease, i.e., causing regression of the disease.

As used herein, the term “a filovirus-mediated disease” encompasses a condition which is a direct result of filovirus infection; and a condition which is an indirect result, e.g., a sequela, of a filovirus infection. Such conditions include, but are not limited to, fever, internal hemorrhaging, edema, organ failure, headache, malaise, myalgia, nausea, vomiting, bleeding of needle puncture sites, hematemesis, melena, petechiae, ecchymosis, maculopapular rash, disseminated intravascular coagulation, shock, jaundice, conjunctivitis, diarrhea, pharyngitis, convulsions, delirium, coma, oligura, and epistaxis.

By “an effective amount” is meant the amount of a compound, alone or in combination with another therapeutic regimen, required to immunize an equine (in the case of immunogens disclosed herein) or to treat a patient with a filovirus-mediated disease (e.g., any virus described herein including an Ebola virus or Marburg virus) in a clinically relevant manner (in the case of equine-derived hyperimmune immunoglobulin compositions as provided herein). A sufficient amount of an active compound used to immunize an equine and/or treat conditions caused by a virus varies depending upon the manner of administration, the age, body weight, and general health of the patient. Ultimately, the prescribers will decide the appropriate amount and dosage regimen.

As used herein, the term “adjuvant” is intended to encompass a substance or vehicle that non-specifically enhances the immune response to an antigen. Adjuvants can include a suspension of minerals (such as alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity.

Chimeric Filovirus Glycoprotein Polypeptides and Polynucleotides

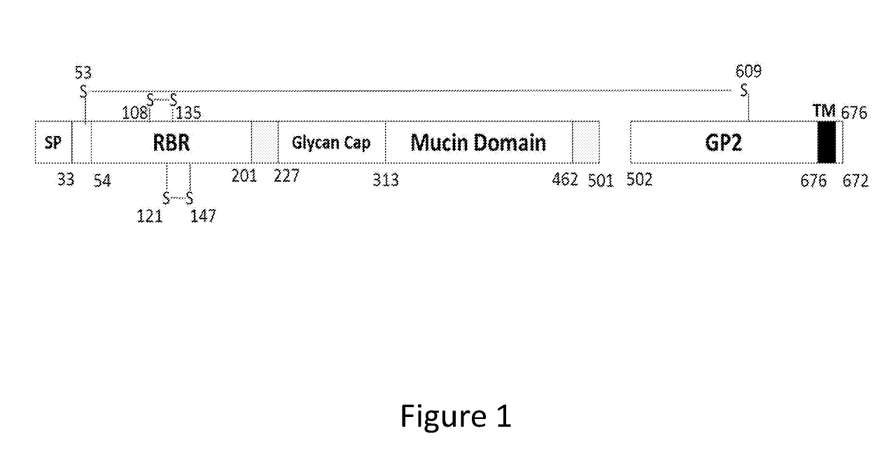

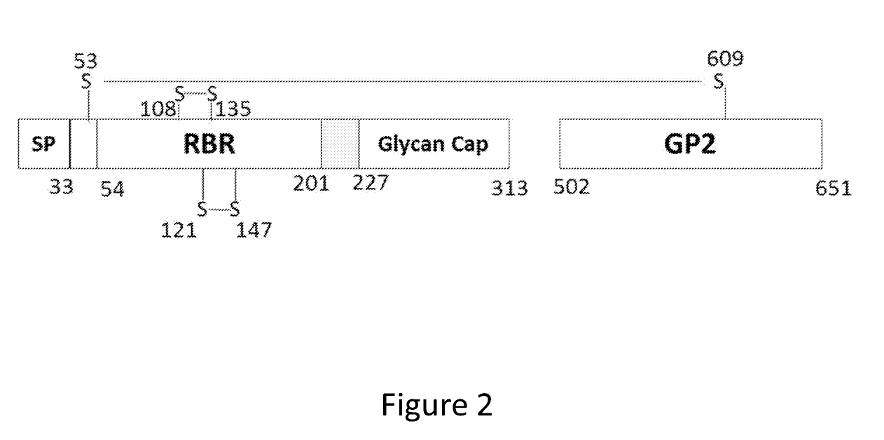

The disclosure provides a chimeric filovirus spike glycoprotein polypeptide. In some embodiments, the chimeric filovirus spike glycoprotein polypeptide comprises a specific region of the MARV or EBOV GP1 of approximately 150 amino acids that was previously shown to bind filovirus receptor-positive cells, but not receptor-negative cells, more efficiently than GP1, and inhibit entry of these respective viruses (Kuhn, J. H. et al. (2006)). This region of glycoprotein is referred to herein as the receptor binding region (RBR) and is part of a larger domain (referred to here as GP-deltaMuc, GP-ΔMuc or GP-dMuc) that excludes the highly glycosylated and bulky mucin-like domain (MLD). FIG. 1 illustrates the domain structure of a typical filovirus glycoprotein. FIG. 2 illustrates the domain structure of a filovirus GP-ΔMuc construct.

In some embodiments, a chimeric filovirus spike glycoprotein polypeptide can comprise an amino acid sequence at least 70%, 75%, 80%, 85%, 90%, 95%, or 100% identical to SEQ ID NO: 2, SEQ ID NO: 4, SEQ ID NO: 6, SEQ ID NO: 8, SEQ ID NO: 10, or SEQ ID NO: 12. The terms “identical” or percent “identity” in the context of two or more amino acid sequences, refer to two or more sequences or subsequences that are the same or have a specified percentage of amino acid residues that are the same, when compared and aligned for maximum correspondence over a comparison window, as measured using one of the following sequence comparison algorithms or by manual alignment and visual inspection.

Optimal alignment of sequences for comparison can be conducted, for example, by a local homology algorithm (Smith & Waterman, Adv. Appl. Math. 2:482 (1981), by a global alignment algorithm (Needleman & Wunsch, I Mol. Biol. 48:443 (1970), by search for similarity methods (Pearson & Lipman, Proc. Natl. Acad. Sci. U.S.A. 85:2444 (1988); Altschul et al., Nucl. Acids Res. 25:3389-402 (1997), by computerized implementations of these algorithms (e.g., GAP, BESTFIT, FASTA, and BLAST in the Wisconsin Genetics Software Package, Genetics Computer Group, 575 Science Dr., Madison, Wis.), typically using the default settings, or by manual alignment and visual inspection (see, e.g., Current Protocols in Molecular Biology, Ausubel et al. (eds.), 1994). For example, BLAST protein searches can be performed using the XBLAST program, score=50, wordlength=3 to obtain amino acid sequences that are more than 80% identical to the amino acid sequence of SEQ ID NOs: 2, 4, 6, 8, 10, or 12.

A chimeric filovirus glycoprotein polypeptide can be expressed using an expression vector and purified. Expression vectors can be either self-replicating extrachromosomal vectors or vectors which integrate into a host genome. Generally, expression vectors include transcriptional and translational regulatory nucleic acid sequences operably linked to the nucleic acid encoding the target protein. The term “control sequences” refers to DNA sequences necessary for the expression of an operably associated coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. Nucleic acid is “operably associated” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Operably associated DNA sequences can be contiguous or non-contiguous. Methods for associating DNA sequences are well-known in the art and include use of the polymerase chain reaction and ligation. The transcriptional and translational regulatory nucleic acid will generally be appropriate to the host cell used to express the target protein; for example, transcriptional and translational regulatory nucleic acid sequences from E. coli are can be used to express the target protein in E. coli.

Numerous types of appropriate expression vectors, and suitable regulatory sequences are known in the art for a variety of host cells. Methods for expressing polypeptides are well known in the art (e.g., Sambrook et al. (1989) Molecular Cloning, A Laboratory Manual, 2nd ed., vol. 1-3, Cold Spring Harbor Laboratory; Berger and Kimmel (1987) Guide to Molecular Cloning Techniques, Methods in Enzymology, vol. 152, Academic Press, Inc., San Diego, Calif.; Ausubel et al. (1995) Current Protocols in Molecular Biology, John Wiley & Sons, Inc., NY)

Chimeric filovirus glycoproteins can be produced by culturing a host cell transformed with an expression vector containing nucleic acid encoding a chimeric filovirus glycoprotein, under the appropriate conditions to induce or cause expression of the polypeptide. The conditions appropriate for protein expression will vary with the choice of the expression vector and the host cell, and will be easily ascertained by one skilled in the art using routine experimentation. For example, the growth and proliferation of the host cell can be optimized for the use of constitutive promoters in the expression vector, and appropriate growth conditions for induction are provided for use of an inducible promoter. In addition, in some embodiments, the timing of the harvest is a factor, for example, when using baculoviral systems. One of skill in the art will recognize that the coding sequences can be optimized for expression in the selected host cells.

Appropriate host cells include yeast, bacteria, archaebacteria, fungi, and insect and animal cells, including mammalian cells. Host cells include, but are not limited to, Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, Hep G2 cells, and human cells and cell lines.

The disclosure further provides an isolated nucleic acid molecule encoding a provided chimeric filovirus glycoproteins. Thus, some embodiments provide an isolated nucleic acid molecule encoding a polypeptide comprising an amino acid sequence selected from the group consisting of SEQ ID NOs: 2, 4, 6, 8, 10, or 12. The term “isolated nucleic acid molecule(s)” refers to a nucleic acid molecule, DNA or RNA, which has been removed from its native environment. For example, recombinant DNA molecules contained in a vector are considered isolated, as well as non-naturally occurring, for the purposes of the present disclosure. Further examples of isolated DNA molecules include recombinant DNA molecules maintained in heterologous host cells or purified (partially or substantially) DNA molecules in solution.

The disclosure further provides a polynucleotide comprising a nucleic acid sequence at least 70%, 75%, 80%, 85%, 90%, 95%, or 100% identical to of SEQ ID NO: 1, SEQ ID NO: 3, SEQ ID NO: 5, SEQ ID NO: 7, SEQ ID NO: 9, or SEQ ID NO: 11. In certain embodiments a polynucleotide can comprise the coding sequence for a provided chimeric filovirus glycoprotein polypeptide fused in the same reading frame to a polynucleotide which aids, for example, in expression and secretion of the polypeptide from a host cell (e.g. a leader sequence which functions as a secretory sequence for controlling transport of a polypeptide from the cell).

In certain embodiments a polynucleotide can comprise the coding sequence for a mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g. COS-7 cells) is used.

In another aspect, a chimeric filovirus glycoprotein polypeptide can be used to induce an immune response to filoviruses in a subject, e.g., a horse or other equine, for the production of immune globulin. An effective amount is sufficient to induce an immune response in the recipient. An immunogenic composition for use in the compositions and methods provided herein can be formulated in a suitable delivery vehicle. For example, one suitable carrier includes saline, which can be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water.

Equine Immunoglobulin Compositions

This disclosure provides a pharmaceutical composition derived from polyclonal immunoglobulin of an equine that has been hyper-immunized with one or more filovirus immunogens, e.g., a filovirus glycoprotein. Immunoglobulin specific for any filovirus, or a mixture of filoviruses, can be produced. For example, the filovirus can be Ebola virus (EBOV), Sudan virus (SUDV), Bundibugyo virus (BDBV), Tai Forrest virus (TAFV), Reston virus (RESTV), Marburg virus (MARV), or any combination thereof. The composition can be in the form of plasma, serum or purified immunoglobulin, e.g., purified IgG or fragments thereof; e.g., Fab, Fab′ and F(ab′)₂ fragments. The composition is prepared by immunizing an equine, e.g., a horse, with one or more filovirus immunogens, e.g., a filovirus virus-like particle (VLP) and/or a filovirus glycoprotein, e.g., a GP-ΔMuc or a GP-ATM as described herein, e.g., a filovirus glycoprotein comprising an amino acid sequence at least 90%, at least 95%, or 100% identical to SEQ ID NOS: 2, 4, 6, 8, 10, or 12.

The equine is immunized so as to mount a potent immune response to the immunogen(s), thereby producing large quantities of anti-filovirus antibodies. Blood, serum, or plasma can be recovered from the immunized equine, e.g., by venipuncture of plasmapheresis, and the immunoglobulin can be purified and/or processed by techniques well known to those of ordinary skill in the art. The recovered immunoglobulin can then be formulated with suitable carriers, excipients, preservatives, and/or other additives to produce a pharmaceutical composition for administration to a subject.

In certain aspects, the equine has been hyper-immunized with filovirus immunogens, e.g., a filovirus virus-like particle (VLP) and/or a filovirus glycoprotein, such that a large portion of the IgG circulating in the blood of the equine is specific for the filovirus. For example in certain aspects at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, or even more of the IgG circulating in the blood of the equine is filovirus-specific. The filovirus specific IgG can be purified to produce a high potency treatment. In certain aspects, the filovirus-specific antibody titers in the recovered serum or plasma can result in an EC50 antibody titer for binding to a filovirus glycoprotein, e.g., an EBOV or MARV GP-ΔTM or GP-ΔMuc, of at least 10², at least 5×10², at least 10³, at least 5×10³, least 10⁴, at least 5×10⁴, least 10⁵, or at least 5×10⁵, at least 10⁶, at least 5×10⁶, at least 10⁷, or at least 5×10⁷, as determined by ELISA.

In certain aspects, an equine immunoglobulin pharmaceutical composition as provided herein, e.g., purified immunoglobulin, e.g., purified IgG can have high potency for a filovirus glycoprotein, e.g., an EBOV or MARV GP-ΔTM or GP-ΔMuc. For example in certain aspects the purified IgG can bind to MARV or EBOV GP-ΔTM or GP-ΔMuc with an EC50 of less than 3 μg/ml, less than 2.5 μg/ml, less than 2 μg/ml, less than 1.5 μg/ml, or less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, less than 0.1 μg/ml, or less than 0.09 μg/ml, as measured ELISA.

In certain aspects an equine immunoglobulin pharmaceutical composition as provided herein, e.g., purified immunoglobulin, e.g., purified IgG can be used to treat a subject infected with a filovirus, or to protect a subject susceptible to being infected with a filovirus. In certain aspects the composition as provided herein can be administered before or after filovirus infection, e.g., within 12, 24, 36, 48, or 60 hours of infection or after detection of symptoms, or even at a later time. In certain aspects, two doses of about 100 mg/kg administered to a mouse following a lethal challenge with a filovirus can protect the mouse against the lethal challenge, e.g., result in a cure, reduce symptoms, prolong survival

In certain aspects, an equine immunoglobulin pharmaceutical composition as provided herein, e.g., purified immunoglobulin, can prevent or minimize symptoms in a subject infected with a filovirus when administered to the subject, e.g., by IV infusion.

In certain aspects, the equine is administered a filovirus glycoprotein immunogen. In certain aspects the immunogen is a mucin-like domain-deleted filovirus spike glycoprotein. In certain aspects the transmembrane domain of the spike glycoprotein is deleted instead of, or in combination with the mucin-like domain deletion. In certain aspects, the immunogen comprises the GP1 subunit or a fragment thereof from MARV, EBOV, SUDV, BDBV, TAFV, RESTV, or a combination of GP1 subunits thereof. In certain aspects, the immunogen can comprise the GP1 subunit or a fragment thereof and the GP2 subunit or a fragment thereof from MARV, EBOV, SUDV, BDBV, TAFV, RESTV, or a combination of GP1 and GP2 subunits thereof. In certain aspects, the immunogen comprises an amino acid sequence that is at least 90%, at least 95%, or 100% identical to SEQ ID NOS: 2, 4, 6, 8, 10, or 12.

In certain aspects, an equine immunoglobulin pharmaceutical composition as provided herein, e.g., purified immunoglobulin, can be produced via multiple immunizations of the equine and regularly spaced intervals, immunization regimens can easily be determined by a person of skill in the art. In one aspect, the equine is immunized with the spike glycoprotein on days 0, 21, 42, and 63, and polyclonal immunoglobulin is recovered as plasma on day 90 via plasmapheresis. In other aspects, the equine is immunized with a VLP, e.g., a VLP comprising a filovirus glycoprotein, a filovirus VP40, and a filovirus nucleoprotein (NP) on days 0 and 21, and then boosted with a filovirus spike glycoprotein on days 42 and 63.

In certain aspects, the equine is immunized in a prime-boost regimen. For example, the immunization regimen can include one, two, or more priming immunizations, e.g., with a VLP, and one, two, or more boosting immunizations, e.g., with a glycoprotein subunit, e.g., GP-ΔMuc, GP-ΔTM, or a combination thereof. In certain aspects, the VLP comprises a filovirus glycoprotein and a filovirus VP40. In certain aspects the VLP further comprises the filovirus nucleoprotein (NP).

In certain aspects the disclosure provides a method for preparing an equine immunoglobulin pharmaceutical composition as provided herein, e.g., purified immunoglobulin, where the method comprises administering a filovirus immunogen, e.g., one, two, three, or more filovirus immunogens to an equine, in an amount sufficient to hyperimmunize the equine against protective antigens of the filovirus. In certain aspects at least one filovirus immunogen comprises a filovirus spike glycoprotein, e.g., GP-ΔMuc, GP-ΔTM, or a combination thereof. The method further comprises recovering immunoglobulin from the equine, e.g., through a blood draw or plasmapheresis. In certain aspects the immunoglobulin is recovered as plasma. In certain aspects the method further comprises purifying the immunoglobulin recovered from the equine. In certain aspects the immunoglobulin comprises IgG or a fragment thereof.

The disclosure further provides a method for preventing, treating, or managing a filovirus-mediated disease in a subject, comprising administering to a subject in need of treatment a composition as described herein comprising polyclonal immunoglobulin from an equine that has been hyper-immunized with a filovirus glycoprotein.

In certain aspects the filovirus-mediated disease comprises one or more symptoms selected from the group consisting of: fever, internal hemorrhaging, edema, organ failure, headache, malaise, myalgia, nausea, vomiting, bleeding of needle puncture sites, hematemesis, melena, petechiae, ecchymosis, maculopapular rash, disseminated intravascular coagulation, shock, jaundice, conjunctivitis, diarrhea, pharyngitis, convulsions, delirium, coma, oligura, and epistaxis. In certain aspects the Filovirus mediated disease is Ebola hemorrhagic fever. In certain aspects, the subject to be treated is a human, e.g., a human infected with Ebola virus.

The practice of the disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevier, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlag (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

### EXAMPLES

### Example 1: Identification of Filovirus Glycoprotein Receptor Binding Region (RBR) and Production of GP-ΔMuc Proteins in Mammalian Cells

The crystal structure of a trimeric, pre-fusion conformation of glycoprotein in complex with a neutralizing antibody, KZ52 has been solved at 3.4 angstroms (Å) (Lee, J. E. et al. Nature 454:177-82 (2008)). In this structure, three GP1 subunits assemble to form a chalice, cradled in a pedestal comprised of the GP2 fusion subunits, while the mucin-like domain (MLD) restricts access to the conserved RBR sequestered in the chalice bowl. Based on this structure the RBR is sequestered in the bowl of the GP chalice, partially masked by the large attached MLD, but could become better exposed after proteolytic remodeling by cathepsin enzymes in the target cell endosome. EBOV, SUDV, and MARV glycoproteins are cleaved by cathepsin proteases as an essential step in entry. Cleavage reduces GP1 to an ˜18 kDa product (Chandran, K., et al. Science; Kaletsky, R. L. et al. J Virol 81:13378-84 (2007); Schornberg, K. et al. J Virol 80:4174-8 (2006)). The structures suggest that the most likely site of cathepsin cleavage is the flexible β13-β14 loop of GP1 and illustrate how cleavage there would release the heavily glycosylated regions from GP, leaving just the core of GP1, encircled by GP2, with the receptor-binding site now well exposed. Biochemical studies on EBOV GP support the notion that cathepsin cleavage enhances attachment, presumably better exposing the RBR for interaction with cell surface factors trafficked with the virus into the endosome (Dube, D. et al. J Virol 83:2883-2891 (2009)). The RBR, however, appears at least partially or transiently exposed on the viral surface, and hence, any antibodies that could be targeted to this site can be therapeutically beneficial.

The MLD probably dominates host-interaction surfaces of filovirus GP, and indeed, antibodies against the MLD have been frequently identified. The seclusion of the RBR in the full length GP and its exposure upon cathepsin cleavage during entry suggest that an antigen lacking the bulky MLD would expose a vulnerable of portion of GP to the immune system. Therefore, the ability of such deletion mutant (GP-ΔMuc) to act as a potential pan-filovirus vaccine capable of providing broad protection among various Ebola and Marburg strains was examined.

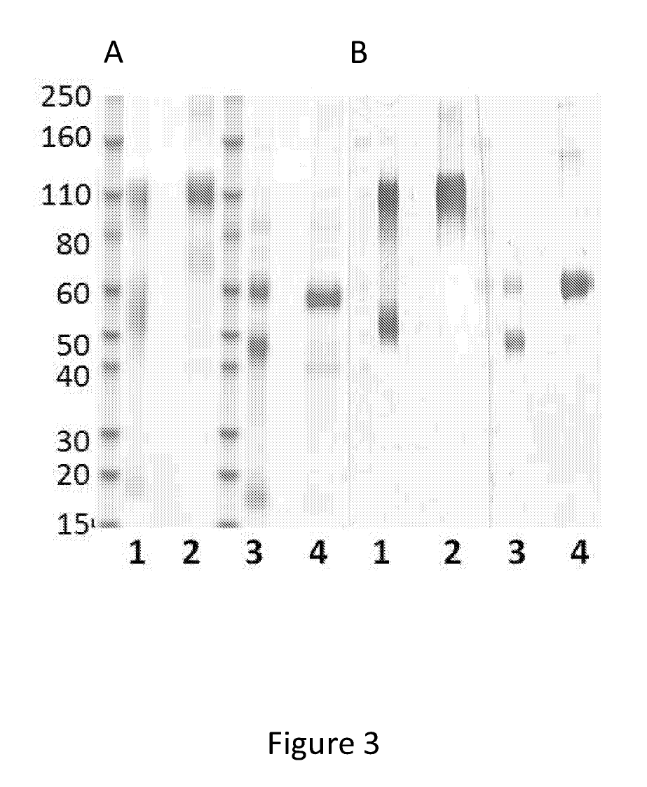

In this example, constructs expressing the amino-terminal subdomain of GP1 (devoid of MLD) and in complex with GP2 devoid of the transmembrane domain (linked through disulfide bonds) were generated. Recombinant GP-ΔMuc (GP without the transmembrane and mucin like domains, SEQ ID NOs 2, 4, and 6) and GP-ΔTM proteins (GP without the transmembrane domain, SEQ ID NOs 8, 10, and 12) from SUDV, EBOV, and MARV were transiently expressed in 293T cells and purified by a multi-step column chromatography method that, dependent on the virus strain, included an anion exchange capture step and size-exclusion chromatography or lectin-affinity resin for further purification of the proteins. FIG. 3 shows SDS-PAGE (A) and a Western Blot (B) of purified EBOV GP-ΔTM and GP-ΔMuc. In the reduced lanes, the top band is GPnull, middle is GP1, and lower is GP2. As shown in FIG. 3B, GP2 is not recognized by antibody used for Western. Lane 1: GP-ΔTM (reducing), Lane 2: GP-ΔTM (non-reducing), Lane 3: GP-ΔMuc (reducing), and Lane 4: GP-AMuc (non-reducing).

Antibody as a reliable marker of protection:

An analysis of sera from vaccination studies performed with an EBOV virus-like particle (VLP) vaccine or an adenovirus vaccine expressing EBOV GP in 81 macaques, to evaluate the relationship between protection from lethal challenge and antibody response to full length GP ectodomain (GP-ΔTM), GP-ΔMuc, and VP40. An increase in antibody levels against GP, but not VP40, was significantly associated with an increased probability of survival following lethal challenge (Table 1).

TABLE 1
Antibody level vs. outcome in 81 vaccinated monkeys

| | | GPdTM titer AU/ml | | GPΔmuc titer AU/ml | | VP 40 | |
|---|---|---|---|---|---|---|---|
| | n | Mean (SD) | Median | Mean (SD) | Median | Mean (SD) | Median |
| Alive | 39 | 1062.4 (881.0)* | 798.1 | 1253.0 (790.2)* | 1218.5 | 4351.2 (4299.7) | 2962 |
| Dead | 42 | 313.7 (342.0) | 168.9 | 541.4 (617.9) | 384.3 | 4855.9 (5159.0) | 3395 |

Most Protective Antibodies Bind to GP-ΔMuc.

The MLD domain is known to mask the core GP structure (Lee, J. E., et al. Nature, 2008. 454(7201): p. 177-82), thus removal of MLD is expected to expose target epitopes for effective neutralization of EBOV. For example, based on structural studies, the mAbs 2G4 and 4G7 (components of ZMapp) binding site maps to the contact points of GP1 and GP2 (Murin, C. D., et al., Proc Natl Acad Sci USA, 2014. 111(48): p. 17182-7), and the 13C6 (another component of ZMapp) binds to a conformational epitope in the glycan Cap (Saphire, E. O., Immunotherapy, 2013. 5(11): p. 1221-33). Thus, the epitopes for all three mAbs included in ZMapp map to non-MLD portions of the GP. The notion of most protective antibodies mapping to GP-ΔMuc is further strengthened by the report of two additional antibodies mapping to the receptor binding region that protected macaques (Marzi, A., et al., PLoS One, 2012. 7(4): p. e36192).

### Example 2: Preparation and Use of Ebola Hyperimmune Equine Plasma and Purified IgG

In this example, high titer, high potency immunoglobulin against EBOV GP was isolated from hyperimmunized horses, purified, and characterized. The goal was to generate an IgG preparation with over 10% anti-GP content. Such a preparation—if administered at a dose of 150-200 mg/kg—would deliver 15-20 mg/kg of anti-GP antibody.

Horses and Immunization

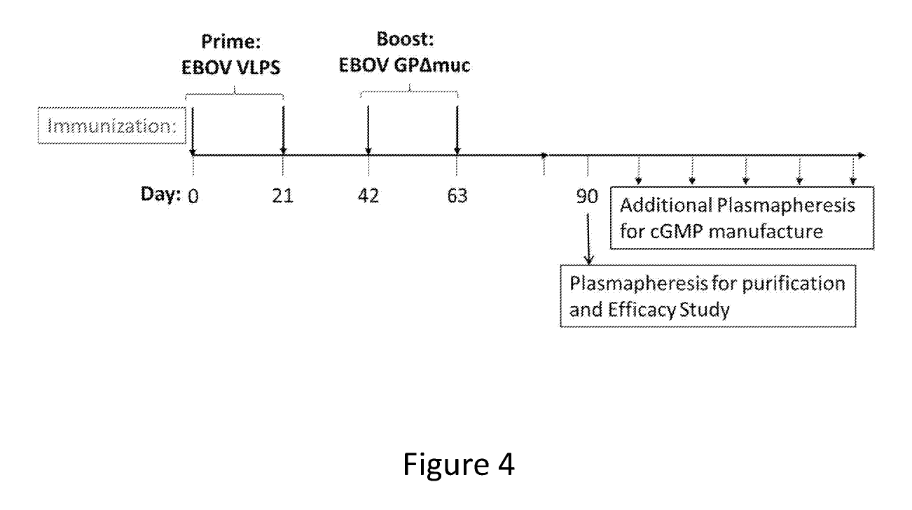

Eight horses (EBOV400, EBOV401, EBOV402, EBOV403, EBOV404, EBOV405, EBOV406, EBOV407) were vaccinated with two priming doses of EBOV VLPs (2 mg of the “triple” VLP comprising GP, VP40, and NP, the first intramuscularly, the second subcutaneously) and two booster doses of EBOV GP-ΔMuc (SEQ ID NO: 4, 250 μg subcutaneously) according to the prime boost schedule shown in FIG. 4. As part of a normal herd health maintenance program, these horses are vaccinated annually against Eastern and Western Equine Encephalitis viruses (killed), West Nile virus (killed), Clostridium tetani toxoid, equine influenza virus (killed), Streptococus equi (modified live or killed), equine herpesviruses (killed), and rabies virus (killed).

Antigens were formulated with Titermax Gold (Sigma) as adjuvant and administered by intramuscular injection on day 0 and subcutaneously on days 21, 42, and 63 in a total volume of 2 ml. Blood samples were obtained from all horses on days 0, 21, 42, 56, and 70 for evaluation of antibody response. A test plasmapheresis on one horse (EBOV401) was performed on study day 90.

Immune Response to EBOV Antigens

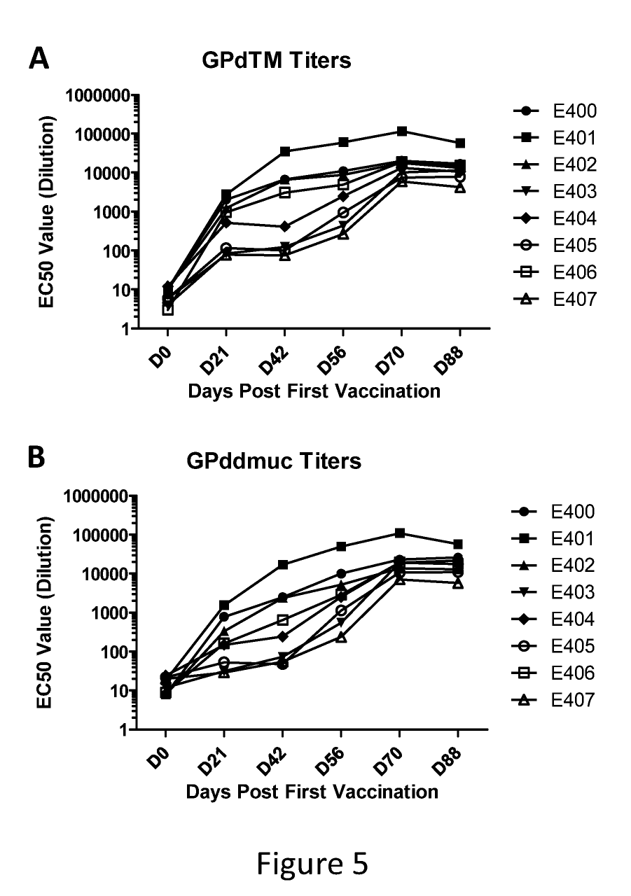

The total IgG response to EBOV GP-ΔTM (lacking only the transmembrane domain) as well as EBOV GP-ΔMuc was evaluated by a quantitative ELISA. Full dilution curve of each sample was used to determine EC₅₀ titers for each plasma sample. As shown in FIG. 5, EC₅₀ titers ranging from 5×10³ to 10⁵ were observed in the immunized horses. These titers compare favorably with the protective titers observed in our previous studies with NHPs (see Table 1).

Purification and Testing of Anti-Ebola Equine IgG

The horse E401 was selected for purification of IgG and preliminary efficacy testing.

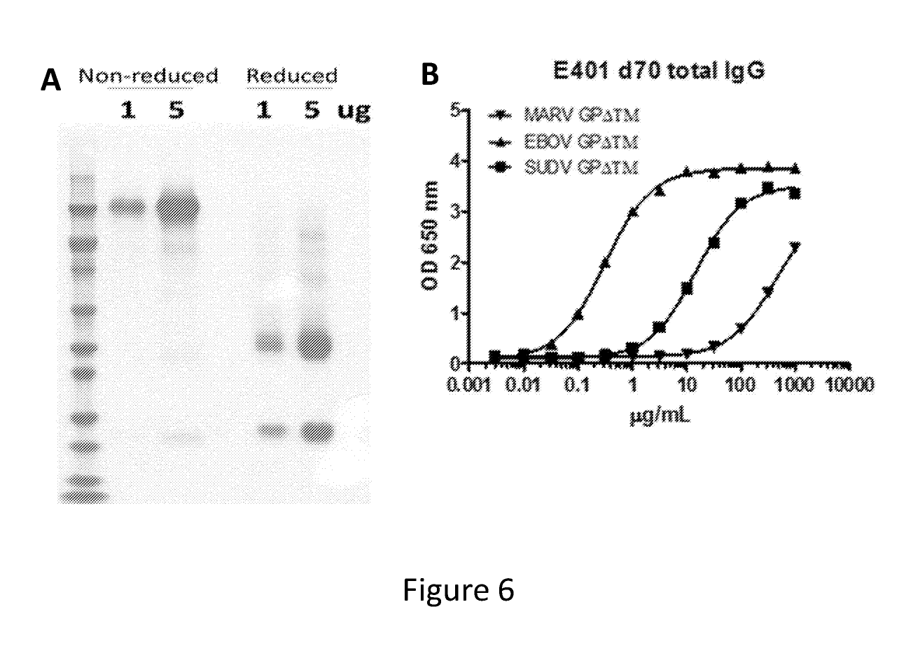

Plasma obtained on day 90 was use to purify IgG by Protein G column. A total of 300 mg IgG (EEIG) was purified and tested by SDS-PAGE and ELISA. FIG. 6 shows the result of the quality control testing for E401 IgG. The purified E401 IgG detected EBOV GP-ΔTM with an EC50 value of 0.306 μg/ml. The E401 equine IgG also cross reacted with SUDV GP with an EC50 of 14.6 μg/ml. MARV GP was detected with an EC50 of 474 ng/ml. The binding to GP-ΔMuc was also tested (data not shown) and the following EC50 values were determined: EBOV (0.308 μg/ml); SUDV (3.05 μg/ml); and MARV (207 μg/ml).

The exact percentage of EBOV specific IgG in these equine preparations was not determined. While not wishing to be bound by theory, based on a conservative assumption of an average EC50 of 50-60 ng/ml for the Ebola specific antibodies, the E401 IgG could have at least 20% Ebola specific antibodies. Based on this conservative estimate, a dose of 100 mg/kg for animal testing would translate into about 20 mg/kg of Ebola specific polyclonal antibodies.

Efficacy Testing of E401 IgG in EBOV Infection Model:

A proof of concept study was performed to assess the ability of EEIG to neutralize EBOV infection in vivo in Balb/c mice model. In this study mice were given EEIG as two intraperitoneal injections of E401 IgG at a dose of 100 mg/kg one hour and 3 days after a lethal challenge with mouse adapted EBOV (1000 PFU). Survival after infection was monitored for 15 days, and will be monitored for 4 weeks. Results from this study showed complete protection of mice from lethal effects of EBOV. The antibody was fully protective at 100 mg/kg dose. Groups of 5 mice were challenged with 1000 PFU of mouse-adapted EBOV. One hour after infection, mice received the first dose of E401-IgG at 100 mg/kg via intraperitoneal (IP) injection. Mice received a second dose of 100 mg/kg E401-IgG on day 3 post infection. Two groups of negative control animals received either no treatment or an irrelevant monoclonal antibody (25 mg/kg). As positive control anti-EBOV monoclonal antibody 6D8 was used at 25 mg/kg (days 0, and 3).

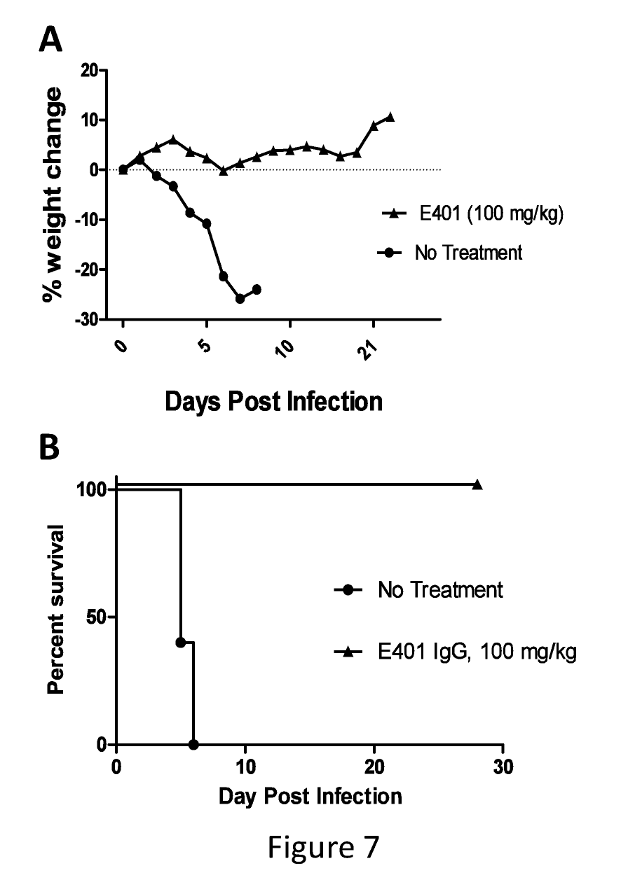

All mice in the control groups succumbed to infection between days 5-7 (FIG. 7) and all 6D8 treated mice survive the challenge (data not shown). Similarly all mice receiving E401-IgG survived the otherwise lethal challenge (FIG. 7).

### Example 3: Preparation and Use of Marburg Equine Immunoglobulin

Horses and Immunization

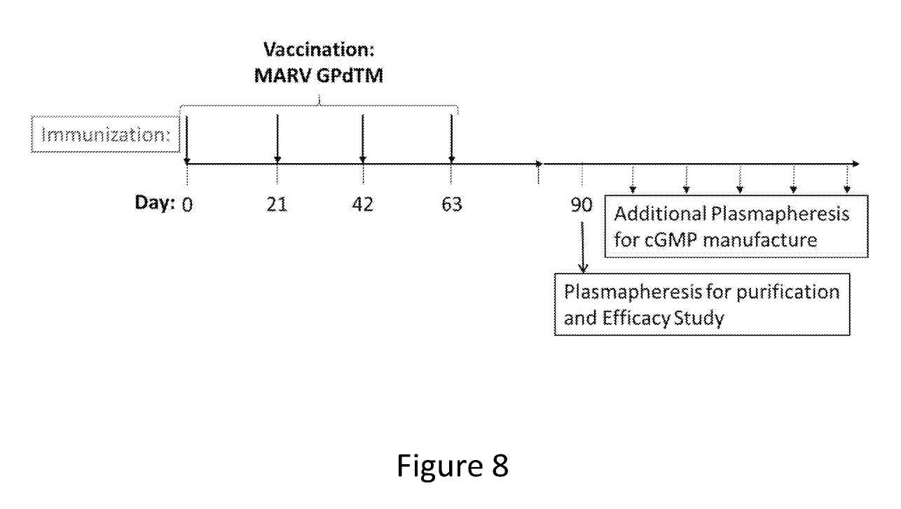

Four horses (designated M300, M304, M305, M306) were immunized with purified Marburg-Angola GP-ΔTM protein (SEQ ID NO: 12), adjuvanted with Titermax Gold according to the schedule shown in FIG. 8.

Immune Response to MARV Antigen

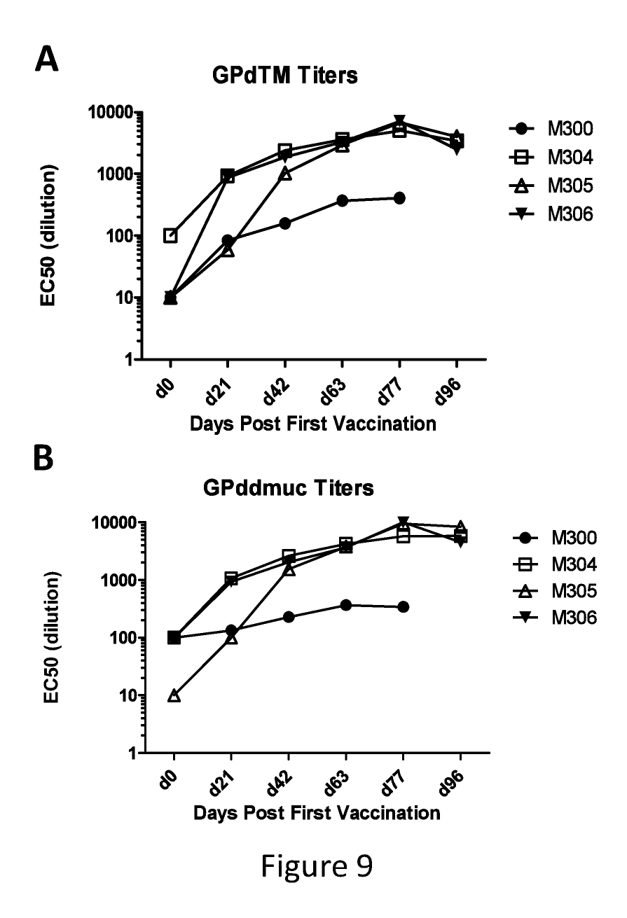

The total IgG response to MARV GP-ΔTM (lacking only the transmembrane domain) as well as MARV GP-ΔMuc was evaluated by a quantitative ELISA. Full dilution curve of each sample was used to determine EC₅₀ titers for reach plasma sample. As shown in FIG. 9, while one of the horses (M300) did not show an appreciable response, the other three horses showed a robust antibody response ranging from 5×10³ to 7×10³ for GP-ΔTM and from 6×10³ to 1×10⁴ for GP-ΔMuc on study day 77. The titers dropped moderately between days 77 and 96. These titers are largely within a favorable range compared to the NHP data shown in Table 1. The titers are below levels obtained for EBOV, due to, for example, the difference in the immunization strategy, i.e. use of prime boost for EBOV, as well as choice of GP antigen type (GP-ΔTM vs. GP-ΔMuc). The horses used in the MARV study had also been previously (˜two years ago) immunized against Marburg in an effort that was abandoned. Initial titers observed in FIG. 96 reflect this preexisting response.

Purification and Testing of Anti-MARV Equine IgG

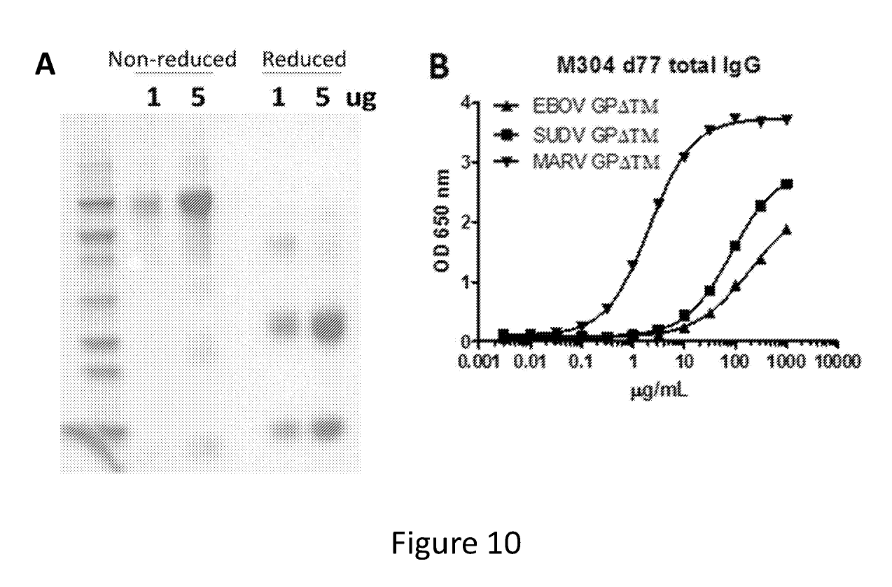

The horses M304-306 were selected for purification of IgG. Plasma collected on day 77 was use to purify IgG by Protein G column. FIG. 10 shows the result of the quality control testing for M304 IgG. The purified M304 IgG detected MARV GP-ΔTM with an EC50 value of 2.11 μg/ml. The EC50 values for all three purified IgGs (in μg/ml) are shown in Table 2. The EC50 values for MARV were in the range of 2 μg/ml.

TABLE 2
ELISA EC50 titers of purified M304-M306 horses IgG on
plates coated with the indicated filovirus glycoproteins.

| | | MARV | MARV | EBOV | EBOV | SUDV | SUDV |
|---|---|---|---|---|---|---|---|
| | Yield (mg) | GPdTM | GPΔmuc | GPdTM | GPΔmuc | GPdTM | GPΔmuc |
| M304 | 243 | 2.11 | 1.12 | 197 | 52.7 | 83.4 | 30.6 |
| M305 | 275 | 1.78 | 0.856 | 43.2 | 112 | 436 | 117 |
| M306 | 299 | 1.99 | 1.06 | 32.7 | 26.1 | 77.1 | 39.1 |

The exact percentage of MARV specific IgG in these equine preparations was not determined. While not wishing to be bound by theory, based on a conservative assumption of an average EC50 of 50-60 ng/ml for the MARV specific antibodies, the IgG purified from these three horses has about 2-3% MARV specific antibodies. Based on this estimate, a dose of 100 mg/kg would translate into about 2-3 mg/kg of MARV specific polyclonal antibodies.

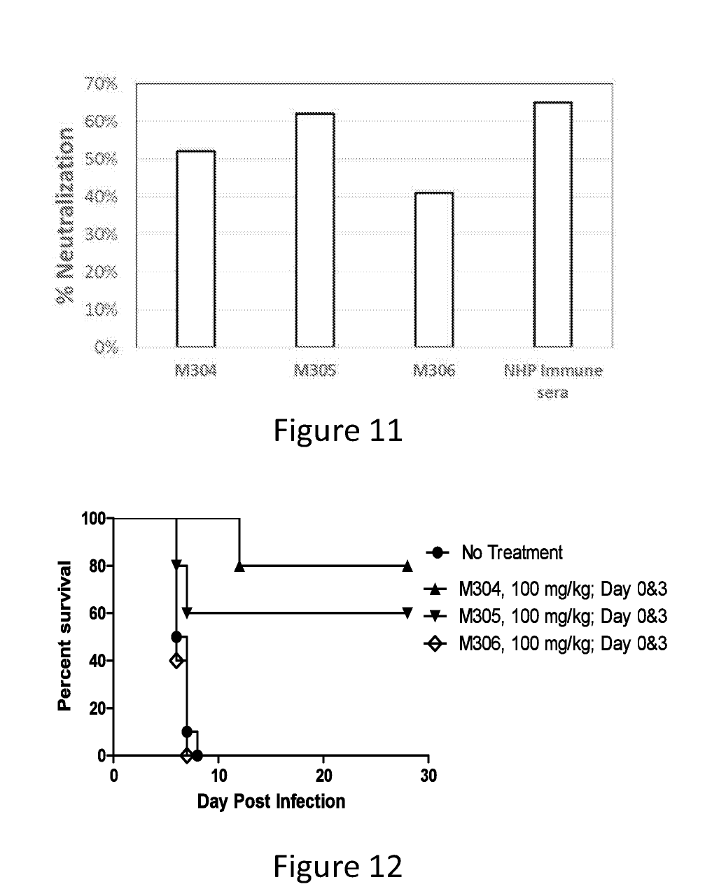

The ability of the purified IgG to neutralize the authentic MARV virus was evaluated using plaque reduction assay. MARV virus was diluted to target ˜150 plaques per well. The virus was preincubated either with PBS or IgG (1 mg/ml) from the MARV immunized horses, or a 1:100 dilution of polyclonal sera from MARV VLP vaccinated NHPs. The virus was then enumerated by standard plaque assay. As shown in FIG. 11, the three IgGs at the indicated concentrations achieved between 40% and 65% neutralization.

Efficacy Testing of Equine Anti-MARV IgG in MARV Infection Model:

Groups of 5 mice were challenged with 1000 PFU of mouse-adapted MARV. One hour after infection mice received the first dose of IgG at 100 mg/kg via intraperitoneal (IP) injection. Mice received a second dose of 100 mg/kg IgG on day 3 post infection. Two groups of negative control animals received either no treatment or an irrelevant monoclonal antibody (25 mg/kg).

All mice in the control groups succumbed to infection between days 6-8 post challenge (FIG. 12). In the M304 treated group, 4 out of 5 mice and in M305 group, 3 out of 5 mice survived the challenge (FIG. 12). In contrast, mice receiving M306 IgG all succumbed to infection (FIG. 12). Thus, IgGs from two of three horses provided partial protection

Future effort on MARV equine IgG development will focus on a prime boost strategy similar to EBOV.

The disclosure is not to be limited in scope by the specific embodiments described which are intended as single illustrations of individual aspects of the disclosure, and any compositions or methods which are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims

All publications and patent applications mentioned in this specification are herein incorporated by reference to the same extent as if each individual publication or patent application was specifically and individually indicated to be incorporated by reference.

SEQUENCES

SEQ ID NO: 1-Sudan Virus Glycoprotein without the Transmembrane and Mucin-like Domains
(SUDV GP-ΔMuc) polynucleotide sequence
ATGGAGGGTCTGTCATTGCTGCAGCTCCCTCGCGATAAGTTCCGTAAAAGCTCATTCTTCGTATGGGTCATAATTCTGTTCCAGAA
GGCGTTCTCTATGCCCCTCGGCGTCGTGACAAACAGTACTTTGGAAGTCACGGAAATAGACCAGCTCGTTTGCAAGGATCATTTGG
CTTCGACAGACCAGCTGAAAAGTGTGGGCCTCAATCTTGAAGGATCGGGCGTGTCGACCGATATCCCCTCCGCTACGAAAAGATGG
GGTTTCCGCAGCGGTGTTCCCCCAAAAGTGGTGAGCTATGAGGCTGGCGAGTGGGCTGAAAACTGTTACAATTTGGAGATTAAGAA
GCCAGATGGATCAGAGTGTCTGCCTCCCCCTCCAGACGGTGTTAGGGGCTTTCCGCGTTGCAGATACGTGCACAAAGCCCAGGGAA
CTGGACCGTGCCCAGGCGACTATGCTTTCCACAAAGACGGCGCCTTTTTCCTCTACGACAGATTGGCGTCTACCGTTATCTACCGT
GGTGTCAACTTCGCTGAGGGAGTAATCGCTTTCCTTATCCTCGCTAAGCCAAAGGAAACTTTCCTCCAAAGCCCGCCTATCCGCGA
AGCGGTCAACTATACCGAGAATACTTCCTCCTACTACGCCACCTCTTACTTGGAGTACGAGATAGAGAACTTCGGTGCACAGCACA
GTACTACACTGTTCAAAATCAATAACAACACTTTTGTGTTGCTGGATAGACCCCATACACCCCAATTCCTTTTCCAGCTCAACGAC
ACGATTCACTTGCACCAACAGTTGTCTAATACAACTGGAAAACTCATTTGGACTCTTGATGCAAACATCAACGCTGACATCGGAGA
ATGGGCCTTTTGGGAGAATAAGAAGAACTTGTCCGAGCAACTGAGGGGTGAGGAACTCTCATTCGAAACCCTTTCGCTTCCCCAAG
AGAGTACCTCGAACGGACTCATCACCTCTACCGTAACTGGCATCCTTGGTTCTCTTGGCCTGCGCAAGAGGTCAAGACGTCAGGTC
AATACCCGCGCTACGGGCAAATGCAACCCGAATCTCCATTACTGGACCGCACAAGAACAGCACAACGCAGCAGGCATCGCCTGGAT
CCCGTATTTTGGACCAGGTGCCGAGGGTATCTACACGGAGGGTTTGATGCACAACCAAAACGCCCTGGTTTGTGGATTGCGCCAAC
TTGCGAACGAAACGACTCAGGCCTTGCAGCTGTTTCTGCGCGCGACTACCGAACTTAGGACATACACAATACTGAACCGTAAGGCC
ATTGACTTCCTGTTGAGAAGGTGGGGTGGTACCTGTCGTATCTTGGGACCAGATTGCTGCATTGAACCTCACGACTGGACGAAGAA
CATTACAGACAAGATAAACCAAATCATCCATGATTTCATCGACAACCCTCTGCCAAATCACCACCACCATCATCATCACCATCACC
ACTGA
SEQ ID NO: 2-Sudan Virus Glycoprotein without the Transmembrane and Mucin-like Domains
(SUDV GP-ΔMuc) polypeptide sequence
M E G L S L L Q L P R D K F R K S S F F V W V I I L F Q K A F S M P L G V V T N S T L
E V T E I D Q L V C K D H L A S T D Q L K S V G L N L E G S G V S T D I P S A T K R W
G F R S G V P P K V V S Y E A G E W A E N C Y N L E I K K P D G S E C L P P P P D G V
R G F P R C R Y V H K A Q G T G P C P G D Y A F H K D G A F F L Y D R L A S T V I Y R
G V N F A E G V I A F L I L A K P K E T F L Q S P P I R E A V N Y T E N T S S Y Y A T
S Y L E Y E I E N F G A Q H S T T L F K I N N N T F V L L D R P H T P Q F L F Q L N D
T I H L H Q Q L S N T T G K L I W T L D A N I N A D I G E W A F W E N K K N L S E Q L
R G E E L S F E T L S L P Q E S T S N G L I T S T V T G I L G S L G L R K R S R R Q V
N T R A T G K C N P N L H Y W T A Q E Q H N A A G I A W I P Y F G P G A E G I Y T E G
L M H N Q N A L V C G L R Q L A N E T T Q A L Q L F L R A T T E L R T Y T I L N R K A
I D F L L R R W G G T C R I L G P D C C I E P H D W T K N I T D K I N Q I I H D F I D
N P L P N H H H H H H H H H H
SEQ ID NO: 3-Ebola Virus Glycoprotein without the Transmembrane and Mucin-like Domains
(EBOV GP-ΔMuc) polynucleotide sequence
ATGGGTGTTACGGGCATACTGCAGCTGCCTCGCGACCGTTTCAAGCGTACGTCTTTCTTCTTGTGGGTCATTATCCTCTTCCAGAG
AACTTTCTCAATTCCACTCGGAGTGATACATAATAGTACTTTGCAAGTTTCCGACGTGGACAAGTTGGTTTGTCGTGATAAACTGT
CATCGACCAACCAACTCCGTTCTGTTGGACTCAACCTCGAAGGCAACGGTGTGGCAACTGACGTCCCATCCGCCACCAAAAGGTGG
GGATTTCGCTCCGGAGTGCCCCCTAAAGTGGTCAATTATGAAGCAGGTGAATGGGCTGAGAATTGCTACAATTTGGAGATCAAGAA
ACCCGACGGATCTGAGTGCCTTCCAGCAGCGCCCGACGGTATCCGTGGCTTCCCACGTTGCCGCTACGTGCACAAGGTCAGTGGCA
CTGGTCCGTGCGCTGGCGACTTCGCTTTCCACAAGGAGGGCGCATTTTTCCTCTACGACCGTCTTGCATCTACCGTCATCTACAGA
GGCACAACGTTCGCTGAGGGTGTGGTAGCGTTCCTGATATTGCCCCAGGCTAAGAAGGACTTCTTCAGTAGCCACCCTTTGCGCGA
GCCTGTCAACGCCACTGAAGATCCTTCATCTGGTTACTACTCGACTACCATTAGGTACCAGGCTACGGGCTTCGGTACCAACGAAA
CCGAGTACCTCTTCGAAGTCGACAACCTTACTTATGTGCAGCTGGAGAGCCGCTTCACGCCCCAGTTTCTCTTGCAGCTGAACGAG
ACAATCTACACCAGCGGAAAGAGGTCCAACACTACCGGTAAACTGATTTGGAAAGTAAACCCTGAAATTGACACTACAATAGGCGA
GTGGGCCTTTTGGGAGACAAAGAAGAATCTGACCAGAAAGATCCGCAGTGAGGAACTGTCATTCACGGTTGTCAACACCCACCATC
AAGATACCGGTGAGGAAAGCGCCAGTTCGGGAAAATTGGGCCTTATTACAAACACAATCGCGGGTGTGGCAGGCCTCATCACCGGA
GGAAGGCGTACAAGGCGCGAGGCGATCGTAAACGCTCAACCTAAGTGCAATCCCAACTTGCACTATTGGACCACTCAAGACGAGGG
TGCCGCTATCGGCCTCGCCTGGATACCCTACTTCGGTCCAGCTGCGGAAGGTATCTATACAGAAGGACTGATGCACAACCAAGATG
GCTTGATCTGCGGTCTGCGCCAACTGGCAAATGAGACAACGCAGGCCTTGCAGCTTTTCCTTAGAGCGACTACCGAACTGAGGACA
TTTTCAATTCTTAATAGAAAGGCCATCGATTTTCTGCTCCAACGCTGGGGTGGAACCTGTCATATACTTGGCCCGGATTGTTGTAT
CGAGCCACACGACTGGACTAAGAACATCACCGACAAGATAGATCAGATCATTCACGATTTCGTCGATAAAACACTGCCTGACCACC
ACCATCACCATCATCATCACCACCATTGA
SEQ ID NO: 4-Ebola Virus Glycoprotein without the Transmembrane and Mucin-like Domains
(EBOV GP-ΔMuc) polypeptide sequence
M G V T G I L Q L P R D R F K R T S F F L W V I I L F Q R T F S I P L G V I H N S T L
Q V S D V D K L V C R D K L S S T N Q L R S V G L N L E G N G V A T D V P S A T K R W
G F R S G V P P K V V N Y E A G E W A E N C Y N L E I K K P D G S E C L P A A P D G I
R G F P R C R Y V H K V S G T G P C A G D F A F H K E G A F F L Y D R L A S T V I Y R
G T T F A E G V V A F L I L P Q A K K D F F S S H P L R E P V N A T E D P S S G Y Y S
T T I R Y Q A T G F G T N E T E Y L F E V D N L T Y V Q L E S R F T P Q F L L Q L N E
T I Y T S G K R S N T T G K L I W K V N P E I D T T I G E W A F W E T K K N L T R K I
R S E E L S F T V V N T H H Q D T G E E S A S S G K L G L I T N T I A G V A G L I T G
G R R T R R E A I V N A Q P K C N P N L H Y W T T Q D E G A A I G L A W I P Y F G P A
A E G I Y T E G L M H N Q D G L I C G L R Q L A N E T T Q A L Q L F L R A T T E L R T
F S I L N R K A I D F L L Q R W G G T C H I L G P D C C I E P H D W T K N I T D K I D
Q I I H D F V D K T L P D H H H H H H H H H H
SEQ ID NO: 5-Angola Marburg Virus Glycoprotein without the Transmembrane and Mucin-
like Domains (MARV-Ang GP-ΔMuc) (heterologous chitinaze leading sequence underlined)
polynucleotide sequence
ATGCCGCTGTACAAACTCTTGAATGTGTTGTGGCTTGTAGCAGTCTCCAATGCGTTGCCCATCCTCGAAATCGCCTCCAACATTCA
GCCACAGAATGTGGACTCCGTGTGCTCTGGAACCTTGCAAAAGACAGAGGACGTCCACCTCATGGGATTCACGCTGTCTGGACAAA
AGGTAGCGGACTCGCCTTTGGAAGCATCTAAGAGGTGGGCTTTTCGTGCTGGAGTACCCCCAAAGAACGTCGAATACACGGAGGGT
GAGGAGGCGAAAACTTGCTACAACATTAGTGTTACCGATCCGAGTGGCAAATCATTGCTTCTTGACCCTCCCACTAATATCCGTGA
TTACCCCAAATGCAAAACTATCCACCACATTCAGGGCCAAAATCCGCATGCTCAGGGAATCGCCCTGCACTTGTGGGGTGCATTCT
TCCTCTATGATCGCATAGCCTCTACAACCATGTACAGAGGTAAAGTGTTCACGGAAGGCAATATCGCCGCTATGATCGTCAACAAG
ACGGTGCATAAGATGATTTTCAGCCGCCAGGGTCAAGGTTACCGCCACATGAATCTGACGTCAACCAACAAGTATTGGACTTCGAG
CAACGGAACACAGACAAACGATACCGGTTGTTTCGGCACTTTGCAAGAATACAATAGTACCAAAAACCAGACGTGCGCCCCATCTA
AAAAGCCCCTCCCGCTTCCTACTGCGCACCCCGAGGTCAAGCTGACTTCCACCTCCACGGACGCTACAAAGCTCAACACTACTGAC
CCAAATAGTGACGATTCCACGGCCCAACACCTGGTTAGCCGTAGAAGGAAGAGGAATATCTTGTGGAGAGAGGGTGACATGTTCCC
TTTCCTCGACGGACTCATCAATGCGCCGATTGACTTTGACCCTGTACCTAATACGAAAACCATCTTCGACGAAAGTTCTAGCTCCG
GTGCCTCAGCCGAGGAAGATCAGCACGCTTCCCCTAACATATCCCTGACTCTGTCCTATTTTCCGAAAGTCAACGAAAATACAGCC
CACTCCGGCGAAAACGAGAATGATTGTGATGCTGAGCTGAGGATCTGGAGCGTCCAAGAGGATGATCTGGCAGCCGGACTCAGTTG
GATACCTTTCTTCGGTCCTGGAATCGAGGGCCTGTACACTGCTGGTTTGATTAAGAACCAGAACAACTTGGTTTGCCGCCTTAGAC
GCCTCGCCAACCAAACGGCAAAGAGTCTGGAGCTTCTCCTCCGCGTGACCACTGAAGAAAGGACATTCAGTCTGATTAACAGACAT
GCCATCGACTTTTTGTTGGCTCGTTGGGGCGGAACTTGTAAGGTCCTCGGACCTGACTGTTGCATCGGTATTGAAGATCTGTCACG
TAATATCAGCGAGCAAATAGACCAAATCAAGAAGGATGAGCAGAAGGAAGGCACCTGA
SEQ ID NO: 6-Angola Marburg Virus Glycoprotein without the Transmembrane and Mucin-
like Domains (MARV-Ang GP-ΔMuc) (heterologous chitinaze leading sequence underlined)
polypeptide sequence
M P L Y K L L N V L W L V A V S N A L P I L E I A S N I Q P Q N V D S V C S G T L Q K
T E D V H L M G F T L S G Q K V A D S P L E A S K R W A F R A G V P P K N V E Y T E G
E E A K T C Y N I S V T D P S G K S L L L D P P T N I R D Y P K C K T I H H I Q G Q N
P H A Q G I A L H L W G A F F L Y D R I A S T T M Y R G K V F T E G N I A A M I V N K
T V H K M I F S R Q G Q G Y R H M N L T S T N K Y W T S S N G T Q T N D T G C F G T L
Q E Y N S T K N Q T C A P S K K P L P L P T A H P E V K L T S T S T D A T K L N T T D
P N S D D S T A Q H L V S R R R K R N I L W R E G D M F P F L D G L I N A P I D F D P
V P N T K T I F D E S S S S G A S A E E D Q H A S P N I S L T L S Y F P K V N E N T A
H S G E N E N D C D A E L R I W S V Q E D D L A A G L S W I P F F G P G I E G L Y T A
G L I K N Q N N L V C R L R R L A N Q T A K S L E L L L R V T T E E R T F S L I N R H
A I D F L L A R W G G T C K V L G P D C C I G I E D L S R N I S E Q I D Q I K K D E Q
K E G T
SEQ ID NO: 7-Sudan Virus Glycoprotein without the Transmembrane Domain (SUDV GP-
ΔTM) polynucleotide sequence
ATGGAGGGATTGTCGCTTTTGCAGTTGCCAAGAGACAAGTTTCGCAAATCGTCCTTTTTCGTCTGGGTTATCATCCTCTTCCAAAA
GGCCTTCTCAATGCCTCTGGGCGTTGTTACCAACTCAACTCTGGAAGTAACCGAGATTGACCAGCTCGTGTGCAAAGACCACTTGG
CCTCCACCGACCAGCTCAAATCAGTTGGACTCAATCTCGAAGGTAGTGGAGTGAGCACAGACATACCAAGTGCGACTAAGAGGTGG
GGTTTCCGTACTGGAGTACCTCCCAAAGTTGTCTCGTACGAAGCTGGCGAATGGGCGGAAAACTGTTACAACTTGGAGATTAAGAA
ACCCGACGGTTCTGAATGCCTCCCGCCTCCGCCTGATGGTGTGCGCGGTTTCCCTCGTTGCAGATATGTGCACAAAGCACAGGGAA
CAGGCCCCTGCCCTGGCGATTACGCTTTCCATAAAGACGGAGCCTTCTTTCTTTATGACCGTTTGGCTAGTACGGTTATCTACAGA
GGTGTCAATTTTGCTGAAGGTGTCATCGCGTTCCTGATCCTGGCTAAGCCGAAGGAGACTTTCCTTCAATCGCCTCCTATCCGTGA
GGCTGTCAATTACACCGAAAACACGAGTTCATACTACGCTACCTCATATCTCGAATACGAGATCGAGAACTTTGGCGCACAGCATT
CTACTACCCTCTTCAAAATCAATAACAATACTTTCGTGCTGCTCGATAGACCGCACACTCCCCAATTCTTGTTTCAACTCAACGAT
ACGATCCATTTGCATCAGCAATTGAGCAATACTACGGGAAAGCTGATTTGGACCTTGGACGCTAACATTAACGCTGATATCGGAGA
GTGGGCATTTTGGGAGAACAAGAAGAATTTGTCCGAGCAGCTGCGCGGTGAAGAACTGTCTTTCGAGACACTGTCTCTGAACGAGA
CAGAGGACGACGACGCCACATCCAGCAGAACAACCAAGGGTCGCATCTCAGACAGGGCCACTAGAAAGTACTCAGATCTGGTCCCC
AAAGACTCTCCGGGAATGGTGTCACTGCACGTGCCCGAAGGCGAAACTACTTTGCCATCTCAAAACTCCACCGAAGGTCGTAGAGT
CGACGTGAACACTCAAGAAACTATTACTGAAACGACCGCGACGATCATAGGCACCAACGGAAACAACATGCAGATCTCGACAATCG
GTACTGGCCTGAGCTCATCCCAAATACTCAGCTCTAGCCCCACAATGGCCCCGAGTCCCGAGACTCAGACTTCTACCACCTATACC
CCCAAGCTCCCAGTGATGACTACTGAAGAATCCACTACCCCACCACGCAACAGTCCTGGCTCCACAACCGAGGCTCCAACCTTGAC
TACTCCAGAAAACATTACTACAGCTGTCAAGACCGTGTTGCCGCAAGAGAGTACATCGAATGGCCTGATAACTTCCACCGTGACCG
GTATTCTTGGTAGTCTCGGCCTCCGTAAGAGAAGCCGTCGCCAAGTAAACACACGCGCAACAGGAAAATGTAACCCTAATTTGCAT
TACTGGACCGCGCAAGAACAGCACAACGCTGCCGGCATCGCGTGGATTCCATACTTCGGTCCCGGTGCCGAGGGAATCTACACTGA
AGGCTTGATGCACAATCAGAACGCACTTGTTTGTGGCCTCCGCCAACTGGCCAATGAGACTACCCAGGCTCTGCAGTTGTTTCTGC
GTGCAACGACGGAACTTAGGACATACACTATATTGAACCGCAAGGCCATTGACTTCTTGCTCAGGCGTTGGGGCGGTACGTGCAGA
ATACTTGGCCCAGACTGTTGCATAGAGCCACACGATTGGACCAAGAACATCACGGATAAGATCAACCAGATCATCCACGATTTCAT
CGATAACCCCCTTCCCAACCACCATCACCATCATCACCACCACCATCACTGA
SEQ ID NO: 8-Sudan Virus Glycoprotein without the Transmembrane Domain (SUDV GP-
ΔTM) polypeptide sequence
M E G L S L L Q L P R D K F R K S S F F V W V I I L F Q K A F S M P L G V V T N S T L
E V T E I D Q L V C K D H L A S T D Q L K S V G L N L E G S G V S T D I P S A T K R W
G F R T G V P P K V V S Y E A G E W A E N C Y N L E I K K P D G S E C L P P P P D G V
R G F P R C R Y V H K A Q G T G P C P G D Y A F H K D G A F F L Y D R L A S T V I Y R
G V N F A E G V I A F L I L A K P K E T F L Q S P P I R E A V N Y T E N T S S Y Y A T
S Y L E Y E I E N F G A Q H S T T L F K I N N N T F V L L D R P H T P Q F L F Q L N D
T I H L H Q Q L S N T T G K L I W T L D A N I N A D I G E W A F W E N K K N L S E Q L
R G E E L S F E T L S L N E T E D D D A T S S R T T K G R I S D R A T R K Y S D L V P
K D S P G M V S L H V P E G E T T L P S Q N S T E G R R V D V N T Q E T I T E T T A T
I I G T N G N N M Q I S T I G T G L S S S Q I L S S S P T M A P S P E T Q T S T T Y T
P K L P V M T T E E S T T P P R N S P G S T T E A P T L T T P E N I T T A V K T V L P
Q E S T S N G L I T S T V T G I L G S L G L R K R S R R Q V N T R A T G K C N P N L H
Y W T A Q E Q H N A A G I A W I P Y F G P G A E G I Y T E G L M H N Q N A L V C G L R
Q L A N E T T Q A L Q L F L R A T T E L R T Y T I L N R K A I D F L L R R W G G T C R
I L G P D C C I E P H D W T K N I T D K I N Q I I H D F I D N P L P N H H H H H H H H
H H
SEQ ID NO: 9-Ebola Virus Glycoprotein without the Transmembrane Domain (EBOV GP-
ΔTM) polynucleotide sequence
ATGGGAGTTACGGGCATATTGCAGCTGCCTAGAGACAGATTCAAGAGGACTTCCTTCTTCTTGTGGGTAATCATCTTGTTCCAGAG
GACTTTTTCAATCCCGTTGGGCGTTATTCATAACTCTACTCTGCAAGTGTCCGATGTCGATAAGCTCGTATGTAGGGACAAGCTCA
GCTCAACAAACCAGTTGCGTTCCGTGGGCCTGAACCTCGAGGGAAATGGAGTAGCCACCGATGTACCCTCTGCAACCAAGAGGTGG
GGCTTCCGCAGCGGTGTCCCTCCCAAAGTCGTGAATTACGAAGCTGGCGAATGGGCCGAGAATTGCTATAACCTCGAGATCAAGAA
GCCGGACGGCTCTGAATGTCTGCCTGCCGCTCCTGACGGTATTCGCGGTTTCCCACGTTGCCGTTACGTGCATAAAGTGTCCGGTA
CGGGACCATGCGCAGGCGACTTTGCGTTCCACAAAGAGGGAGCCTTTTTCCTCTACGACCGCCTGGCTTCGACTGTCATCTATCGT
GGAACCACATTCGCCGAAGGTGTGGTAGCCTTTCTGATCCTGCCACAGGCCAAGAAGGACTTCTTCTCTAGCCACCCACTGAGGGA
ACCAGTCAACGCTACAGAAGATCCCAGCAGTGGTTACTACAGTACTACCATCCGCTACCAAGCGACCGGTTTCGGAACGAATGAGA
CTGAGTATCTCTTTGAGGTGGATAACCTGACCTACGTGCAACTCGAATCCAGATTCACGCCTCAATTTCTTCTCCAATTGAACGAG
ACTATCTACACTTCCGGAAAACGTAGTAACACTACGGGAAAGCTGATTTGGAAGGTGAACCCCGAGATCGATACGACTATTGGCGA
GTGGGCCTTTTGGGAAACAAAGAAAAACTTGACGAGGAAGATTCGCTCTGAAGAACTGTCCTTCACTGTCGTGTCAAACGGAGCGA
AAAACATTTCGGGCCAGAGTCCAGCCCGCACCTCGAGTGATCCCGGTACCAATACAACTACAGAGGACCATAAGATCATGGCTTCA
GAGAACTCCTCGGCCATGGTCCAAGTCCACAGTCAAGGACGCGAAGCTGCAGTTAGCCACCTGACTACACTGGCGACCATAAGCAC
ATCTCCGCAGAGCCTTACGACAAAACCTGGTCCCGACAACTCTACGCATAATACGCCCGTGTACAAACTCGATATTAGCGAAGCCA
CCCAAGTCGAACAGCACCACCGTAGAACTGACAACGACTCAACTGCAAGTGATACCCCTTCGGCCACCACAGCTGCTGGCCCACCT
AAAGCTGAGAACACCAATACTAGCAAATCGACCGATTTCCTCGACCCTGCTACAACCACATCACCTCAGAATCACTCCGAAACAGC
GGGTAACAATAACACCCACCATCAAGACACGGGCGAGGAATCTGCCTCTTCCGGCAAACTCGGTCTTATCACAAACACTATCGCGG
GTGTCGCTGGATTGATCACCGGTGGTAGGCGCACTCGTAGAGAGGCAATCGTTAATGCCCAGCCAAAGTGTAACCCCAACTTGCAT
TATTGGACTACTCAAGATGAGGGAGCAGCTATAGGACTCGCTTGGATCCCTTACTTCGGACCCGCTGCAGAGGGCATCTACACGGA
AGGTTTGATGCACAACCAGGACGGCCTTATTTGCGGTCTGAGGCAGCTTGCTAACGAGACAACGCAAGCCCTCCAACTGTTCCTCC
GCGCGACGACGGAGTTGCGCACCTTCAGTATTCTCAACAGGAAGGCTATTGATTTCTTGTTGCAGCGCTGGGGTGGTACCTGCCAC
ATCTTGGGCCCAGACTGTTGCATCGAGCCCCACGATTGGACTAAGAACATTACCGACAAGATCGACCAGATCATCCACGATTTTGT
TGACAAGACGTTGCCAGACCATCACCATCATCATCACCACCACCATCACTGA
SEQ ID NO: 10-Ebola Virus Glycoprotein without the Transmembrane Domain (EBOV GP-
ΔTM) polypeptide sequence
M G V T G I L Q L P R D R F K R T S F F L W V I I L F Q R T F S I P L G V I H N S T L
Q V S D V D K L V C R D K L S S T N Q L R S V G L N L E G N G V A T D V P S A T K R W
G F R S G V P P K V V N Y E A G E W A E N C Y N L E I K K P D G S E C L P A A P D G I
R G F P R C R Y V H K V S G T G P C A G D F A F H K E G A F F L Y D R L A S T V I Y R
G T T F A E G V V A F L I L P Q A K K D F F S S H P L R E P V N A T E D P S S G Y Y S
T T I R Y Q A T G F G T N E T E Y L F E V D N L T Y V Q L E S R F T P Q F L L Q L N E
T I Y T S G K R S N T T G K L I W K V N P E I D T T I G E W A F W E T K K N L T R K I
R S E E L S F T V V S N G A K N I S G Q S P A R T S S D P G T N T T T E D H K I M A S
E N S S A M V Q V H S Q G R E A A V S H L T T L A T I S T S P Q S L T T K P G P D N S
T H N T P V Y K L D I S E A T Q V E Q H H R R T D N D S T A S D T P S A T T A A G P P
K A E N T N T S K S T D F L D P A T T T S P Q N H S E T A G N N N T H H Q D T G E E S
A S S G K L G L I T N T I A G V A G L I T G G R R T R R E A I V N A Q P K C N P N L H
Y W T T Q D E G A A I G L A W I P Y F G P A A E G I Y T E G L M H N Q D G L I C G L R
Q L A N E T T Q A L Q L F L R A T T E L R T F S I L N R K A I D F L L Q R W G G T C H
I L G P D C C I E P H D W T K N I T D K I D Q I I H D F V D K T L P D H H H H H H H H
H H
SEQ ID NO: 11-Angola Marburg Virus Glycoprotein without the Transmembrane Domain
(MARV-Ang GP-ΔTM) (heterologous chitinaze leading sequence underlined)
polynucleotide sequence
ATGCCGCTGTACAAACTCTTGAATGTGTTGTGGCTTGTAGCAGTCTCCAATGCGTTGCCCATCCTCGAAATCGCCTCCAACATTCA
GCCACAGAATGTGGACTCCGTGTGCTCTGGAACCTTGCAAAAGACAGAGGACGTCCACCTCATGGGATTCACGCTGTCTGGACAAA
AGGTAGCGGACTCGCCTTTGGAAGCATCTAAGAGGTGGGCTTTTCGTGCTGGAGTACCCCCAAAGAACGTCGAATACACGGAGGGT
GAGGAGGCGAAAACTTGCTACAACATTAGTGTTACCGATCCGAGTGGCAAATCATTGCTTCTTGACCCTCCCACTAATATCCGTGA
TTACCCCAAATGCAAAACTATCCACCACATTCAGGGCCAAAATCCGCATGCTCAGGGAATCGCCCTGCACTTGTGGGGTGCATTCT
TCCTCTATGATCGCATAGCCTCTACAACCATGTACAGAGGTAAAGTGTTCACGGAAGGCAATATCGCCGCTATGATCGTCAACAAG
ACGGTGCATAAGATGATTTTCAGCCGCCAGGGTCAAGGTTACCGCCACATGAATCTGACGTCAACCAACAAGTATTGGACTTCGAG
CAACGGAACACAGACAAACGATACCGGTTGTTTCGGCACTTTGCAAGAATACAATAGTACCAAAAACCAGACGTGCGCCCCATCTA
AAAAGCCCCTCCCGCTTCCTACTGCGCACCCCGAGGTCAAGCTGACTTCCACCTCCACGGACGCTACAAAGCTCAACACTACTGAC
CCAAATAGTGACGATGAGGACTTGACTACATCGGGTTCGGGCTCTGGTGAGCAGGAGCCCTATACCACGAGCGACGCTGCCACCAA
ACAAGGCCTGAGCTCAACCATGCCCCCTACTCCATCACCTCAACCCTCAACCCCTCAGCAGGGAGGTAACAACACGAACCATAGTC
AAGGCGTGGTTACCGAACCCGGAAAGACCAACACAACCGCCCAGCCAAGTATGCCACCGCATAACACGACCACGATTTCTACAAAC
AACACCTCCAAACACAACCTTAGCACTCCCTCCGTTCCAATCCAGAACGCCACGAACTACAATACTCAATCTACAGCTCCAGAAAA
CGAACAGACCTCTGCACCAAGTAAGACTACCCTTCTCCCTACGGAGAACCCCACCACCGCAAAGTCCACTAACTCGACAAAGTCGC
CCACAACGACTGTGCCAAATACCACAAACAAGTACTCAACTTCTCCCTCTCCGACACCTAACTCCACGGCCCAACACCTGGTTAGC
CGTAGAAGGAAGAGGAATATCTTGTGGAGAGAGGGTGACATGTTCCCTTTCCTCGACGGACTCATCAATGCGCCGATTGACTTTGA
CCCTGTACCTAATACGAAAACCATCTTCGACGAAAGTTCTAGCTCCGGTGCCTCAGCCGAGGAAGATCAGCACGCTTCCCCTAACA
TATCCCTGACTCTGTCCTATTTTCCGAAAGTCAACGAAAATACAGCCCACTCCGGCGAAAACGAGAATGATTGTGATGCTGAGCTG
AGGATCTGGAGCGTCCAAGAGGATGATCTGGCAGCCGGACTCAGTTGGATACCTTTCTTCGGTCCTGGAATCGAGGGCCTGTACAC
TGCTGGTTTGATTAAGAACCAGAACAACTTGGTTTGCCGCCTTAGACGCCTCGCCAACCAAACGGCAAAGAGTCTGGAGCTTCTCC
TCCGCGTGACCACTGAAGAAAGGACATTCAGTCTGATTAACAGACATGCCATCGACTTTTTGTTGGCTCGTTGGGGCGGAACTTGT
AAGGTCCTCGGACCTGACTGTTGCATCGGTATTGAAGATCTGTCACGTAATATCAGCGAGCAAATAGACCAAATCAAGAAGGATGA
GCAGAAGGAAGGCACCTGATAAGCTT
SEQ ID NO: 12-Angola Marburg Virus Glycoprotein without the Transmembrane Domain
(MARV-Ang GP-ΔTM) (heterologous chitinaze leading sequence underlined)
polypeptide sequence
M P L Y K L L N V L W L V A V S N A L P I L E I A S N I Q P Q N V D S V C S G T L Q K
T E D V H L M G F T L S G Q K V A D S P L E A S K R W A F R A G V P P K N V E Y T E G
E E A K T C Y N I S V T D P S G K S L L L D P P T N I R D Y P K C K T I H H I Q G Q N
P H A Q G I A L H L W G A F F L Y D R I A S T T M Y R G K V F T E G N I A A M I V N K
T V H K M I F S R Q G Q G Y R H M N L T S T N K Y W T S S N G T Q T N D T G C F G T L
Q E Y N S T K N Q T C A P S K K P L P L P T A H P E V K L T S T S T D A T K L N T T D
P N S D D E D L T T S G S G S G E Q E P Y T T S D A A T K Q G L S S T M P P T P S P Q
P S T P Q Q G G N N T N H S Q G V V T E P G K T N T T A Q P S M P P H N T T T I S T N
N T S K H N L S T P S V P I Q N A T N Y N T Q S T A P E N E Q T S A P S K T T L L P T
E N P T T A K S T N S T K S P T T T V P N T T N K Y S T S P S P T P N S T A Q H L V S
R R R K R N I L W R E G D M F P F L D G L I N A P I D F D P V P N T K T I F D E S S S
S G A S A E E D Q H A S P N I S L T L S Y F P K V N E N T A H S G E N E N D C D A E L
R I W S V Q E D D L A A G L S W I P F F G P G I E G L Y T A G L I K N Q N N L V C R L
R R L A N Q T A K S L E L L L R V T T E E R T F S L I N R H A I D F L L A R W G G T C
K V L G P D C C I G I E D L S R N I S E Q I D Q I K K D E Q K E G T

What is claimed is:

1. A pharmaceutical composition comprising polyclonal immunoglobulin from an equine that has been hyper-immunized with a filovirus glycoprotein.

2. The composition of claim 1, wherein the immunoglobulin is purified from serum or plasma of the equine that has been hyper-immunized with the filovirus glycoprotein.

3. The composition of claim 2, wherein the purified immunoglobulin is IgG, or a fragment thereof.

4. The composition of claim 3, wherein at least 1%, 2%, 3%, 4%, 5%, 6%, 7%, 8%, 9%, 10%, 11%, 12%, 13%, 14%, 15%, 20%, or more of the purified IgG binds to the filovirus glycoprotein.

5. The composition of any one of claims 2 to 4, wherein the purified immunoglobulin can prevent or minimize symptoms in a subject infected with a filovirus.

6. The composition of any one of claims 1 to 5, wherein the filovirus is Ebola virus (EBOV), Sudan virus (SUDV), Bundibugyo virus (BDBV), Tai Forrest virus (TAFV), Reston virus (RESTV), or Marburg virus (MARV).

7. The composition of any one of claims 1 to 6, wherein the equine is immunized with a mucin-like domain-deleted filovirus spike glycoprotein.

8. The composition of any one of claims 1 to 7, wherein the transmembrane domain of the spike glycoprotein is deleted.

9. The composition of claim 7 or claim 8, wherein the spike glycoprotein comprises the GP1 subunit or a fragment thereof from MARV, EBOV, SUDV, BDBV, TAFV, RESTV, or a combination of GP1 subunits thereof.

10. The composition of claim 7 or claim 8, wherein the spike glycoprotein comprises the GP1 subunit or a fragment thereof and the GP2 subunit or a fragment thereof from MARV, EBOV, SUDV, BDBV, TAFV, RESTV, or a combination of GP1 and GP2 subunits thereof.

11. The composition of any one of claims 7 to 10, wherein the spike glycoprotein comprises an amino acid sequence that is at least 90%, at least 95%, or 100% identical to SEQ ID NOS: 2, 4, 6, 8, 10, or 12.

12. The composition of any one of claims 7 to 11, wherein the equine is immunized with the spike glycoprotein on days 0, 21, 42, and 63.

13. The composition of claim 12, wherein the polyclonal immunoglobulin is recovered as plasma on day 90 via plasmapheresis.

14. The composition of claim 12 or claim 13, wherein the immunogen comprises MARV GP-ATM, and wherein the recovered plasma has an EC50 titer for binding to MARV GP-ATM of at least 10², at least 5×10², at least 10³, at least 5×10³, least 10⁴, at least 5×10⁴, least 10⁵, or at least 5×10⁵, as determined by ELISA.

15. The composition of claim 13 or claim 14, wherein the immunogen comprises MARV GP-ATM, and wherein the purified IgG binds to MARV GP-ΔTM with an EC50 of less than 3 μg/ml, less than 2.5 μg/ml, less than 2 μg/ml, less than 1.5 μg/ml, or less than 1 μg/ml, or less than 0.5 μg/ml, as measured ELISA.

16. The composition of any one of claims 12 to 15, wherein two doses of about 100 mg/kg administered to a mouse following a lethal challenge with a filovirus can protect the mouse against the lethal challenge.

17. The composition of any one of claims 7 to 11, wherein the equine is immunized in a prime-boost regimen.

18. The composition of claim 17, wherein the prime-boost regimen comprises priming with a filovirus virus-like particle (VLP) and boosting with the spike glycoprotein.

19. The composition of claim 18, wherein the VLP comprises a filovirus glycoprotein and a filovirus VP40.

20. The composition of claim 19, wherein the VLP further comprises the filovirus nucleoprotein (NP).

21. The composition of claim 18, wherein the prime dose is administered on day zero and day 21, and the boost dose is administered on day 42 and day 63.

22. The composition of claim 21, wherein the polyclonal immunoglobulin is recovered as plasma on day 90 via plasmapheresis.

23. The composition of any one of claims 17 to 22, wherein the priming immunogen comprises an EBOV VLP comprising GP, VP40, and NP, the boosting immunogen comprises EBOV GP-ΔMuc, and wherein the recovered plasma has an EC50 titer for binding to EBOV GP-ΔTM of at least 10³, at least 5×10³, least 10⁴, at least 5×10⁴, least 10⁵, or at least 5×10⁵, as determined by ELISA.

24. The composition of any one of claims 17 to 23, wherein the priming immunogen comprises an EBOV VLP comprising GP, VP40, and NP, the boosting immunogen comprises EBOV GP-ΔMuc, and wherein the purified IgG binds to EBOV GP-ΔTM or EBOV GP-ΔMuc with an EC50 of less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, less than 0.1 μg/ml, or less than 0.09 μg/ml, as measured ELISA.

25. The composition of any one of claims 17 to 24, wherein two doses of about 100 mg/kg administered to a mouse following a lethal challenge with a filovirus can protect the mouse against the lethal challenge.

26. A method for preparing the composition of any one of claims 1 to 25, comprising
(a) administering an amount of a filovirus immunogen to an equine sufficient to hyperimmunize the equine against protective antigens of the filovirus, wherein the immunogen comprises a filovirus spike glycoprotein; and
(b) recovering immunoglobulin from the equine.

27. The method of claim 26, wherein the immunoglobulin is recovered as plasma.

28. The method of claim 26 or claim 27, further comprising purifying the immunoglobulin recovered from the equine.

29. The method of claim 28, wherein the purified immunoglobulin comprises IgG or a fragment thereof.

30. A method for preventing, treating, or managing a filovirus-mediated disease in a subject, comprising administering to a subject in need of treatment a polyclonal immunoglobulin from an equine that has been hyper-immunized with a filovirus glycoprotein.

31. The method of claim 30, wherein the immunoglobulin is purified from serum or plasma of the equine that has been hyper-immunized with the filovirus glycoprotein.

32. The method of claim 31, wherein the purified immunoglobulin is IgG, or a fragment thereof.

33. The method of claim 32, wherein at least 1%, 2%, 3%, 4%, 5%, 6%, 7%, 8%, 9%, 10%, 11%, 12%, 13%, 14%, 15%, 20%, or more of the purified IgG binds to the filovirus glycoprotein.

34. The method of any one of claims 31 to 33, wherein the purified immunoglobulin can prevent or minimize symptoms in a subject infected with a filovirus.

35. The method of any one of claims 30 to 34, wherein the filovirus is MARV, EBOV, SUDV, BDBV, TAFV, or RESTV.

36. The method of any one of claims 30 to 35, wherein the equine is immunized with a mucin-like domain-deleted filovirus spike glycoprotein.

37. The method of any one of claims 30 to 36, wherein the transmembrane domain of the spike glycoprotein is deleted.

38. The method of claim 36 or claim 37, wherein the spike glycoprotein comprises the GP1 subunit or a fragment thereof from MARV, EBOV, SUDV, BDBV, TAFV, RESTV, or a combination of GP1 subunits thereof.

39. The method of claim 36 or claim 37, wherein the spike glycoprotein comprises the GP1 subunit or a fragment thereof and the GP2 subunit or a fragment thereof from MARV, EBOV, SUDV, BDBV, TAFV, RESTV, or a combination of GP1 and GP2 subunits thereof.

40. The method of any one of claims 36 to 39, wherein the spike glycoprotein comprises an amino acid sequence that is at least 90%, at least 95%, or 100% identical to SEQ ID NOS: 2, 4, 6, 8, 10, or 12.

41. The method of any one of claims 36 to 40, wherein the equine is immunized with the spike glycoprotein on days 0, 21, 42, and 63.

42. The method of claim 40 or claim 41, wherein the polyclonal immunoglobulin is recovered as plasma on day 90 via plasmapheresis.

43. The method of claim 42, wherein the immunogen comprises MARV GP-ΔTM, and wherein the recovered plasma has an EC50 titer for binding to MARV GP-ΔTM of at least 10², at least 5×10², at least 10³, at least 5×10³, least 10⁴, at least 5×10⁴, least 10⁵, or at least 5×10⁵, as determined by ELISA.

44. The method of claim 42 or claim 43, wherein the immunogen comprises MARV GP-ATM, and wherein the purified IgG binds to MARV GP-ΔTM with an EC50 of less than 3 μg/ml, less than 2.5 μg/ml, less than 2 μg/ml, less than 1.5 μg/ml, or less than 1 μg/ml, or less than 0.5 μg/ml, as measured ELISA.

45. The method of any one of claims 42 to 44, wherein two doses of about 100 mg/kg administered to a mouse following a lethal challenge with a filovirus can protect the mouse against the lethal challenge.

46. The method of any one of claims 36 to 40, wherein the equine is immunized in a prime-boost regimen.

47. The method of claim 46, wherein the prime-boost regimen comprises priming with a filovirus VLP and boosting with the spike glycoprotein.

48. The method of claim 47, wherein the VLP comprises a filovirus glycoprotein and a filovirus VP40.

49. The method of claim 48, wherein the VLP further comprises the filovirus nucleoprotein (NP).

50. The method of any one of claims 46 to 49, wherein the prime dose is administered on day zero and day 21, and the boost dose is administered on day 42 and day 63.

51. The method of any one of claims 46 to 50 wherein the polyclonal immunoglobulin is recovered as plasma on day 90 via plasmapheresis.

52. The method of claim 50 or claim 51, wherein the priming immunogen comprises an EBOV VLP comprising GP, VP40, and NP, the boosting immunogen comprises EBOV GP-ΔMuc, and wherein the recovered plasma has an EC50 titer for binding to EBOV GP-ATM of at least 10³, at least 5×10³, least 10⁴, at least 5×10⁴, least 10⁵, or at least 5×10⁵, as determined by ELISA.

53. The method of claim 50 or claim 51, wherein the priming immunogen comprises an EBOV VLP comprising GP, VP40, and NP, the boosting immunogen comprises EBOV GP-ΔMuc, and wherein the purified IgG binds to EBOV GP-ΔTM or EBOV GP-ΔMuc with an EC50 of less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, less than 0.1 μg/ml, or less than 0.09 μg/ml, as measured ELISA.

54. The method of any one of claims 46 to 53, wherein two doses of about 100 mg/kg administered to a mouse following a lethal challenge with a filovirus can protect the mouse against the lethal challenge.

55. The method of any one of claims 30 to 54, wherein the filovirus-mediated disease comprises one or more symptoms selected from the group consisting of: fever, internal hemorrhaging, edema, organ failure, headache, malaise, myalgia, nausea, vomiting, bleeding of needle puncture sites, hematemesis, melena, petechiae, ecchymosis, maculopapular rash, disseminated intravascular coagulation, shock, jaundice, conjunctivitis, diarrhea, pharyngitis, convulsions, delirium, coma, oligura, and epistaxis.

56. The method of any one of claims 30 to 55, wherein the subject is a human.
